(19) United States
(12) Patent Application Publication
(10) Pub. No.: US 2018/0313819 A1
(43) Pub. Date: Nov. 1, 2018

(54) HIGH SPEED DROPLET SORTER

(71) Applicants: Mike Joseph Pugia, Ganger, IN (US); Zane Baird, Brigham City, UT (US); Zehui Cao, Carmel, IN (US)

(72) Inventors: Mike Joseph Pugia, Ganger, IN (US); Zane Baird, Brigham City, UT (US); Zehui Cao, Carmel, IN (US)

(21) Appl. No.: 15/951,822

(22) Filed: Apr. 12, 2018

Related U.S. Application Data
(60) Provisional application No. 62/490,074, filed on Apr. 26, 2017.

(51) Int. Cl. G01N 33/50

(52) U.S. Cl. CPC G01N 33/5005

(57) ABSTRACT

The invention provides a means of generation of high numbers of droplets containing content while still diluting the interfering molecules to allow generating large arrays of isolated droplets without increased need to sort more droplets while allowing a higher content. The invention generates a larger group of droplets containing a library of compounds, then removes the empty droplets and retain the contents of full droplets by size exclusion filtration.

This application claims the priority benefit under 35 U.S.C. section 119 of U.S. Provisional Patent Application No. 62/490,074 entitled “High Speed Droplet Sorter” filed on Apr. 26, 2017; and which is in its entirety herein incorporated by reference.

### BACKGROUND

The invention relates to methods for enriching and detecting rare molecules relative to non-rare molecules. In some aspects the invention relates to methods, apparatus and kits for detecting one or more different populations of rare molecules in a sample suspected of containing the one or more different populations of rare molecules and non-rare molecules. In other aspects, the invention relates to methods and kits for detecting one or more different populations of rare molecules that are freely circulating in samples. In still other aspects, the invention relates to assays, methods and kits for detecting one or more different populations of rare molecules that are associated with rare cells in a sample suspected of containing the one or more different populations of rare cells and non-rare cells.

Encapsulation of cells, biologicals and macromolecules by micro bubbles was proposed by Thomas Chang in 1964, when he introduced the term “artificial cells”. Since then many materials have used to the encapsulate cells, biologicals and macromolecules. Materials such as emsulifiers, oils, surfactants and polymer affect biocompatibility, permeability, mechanical strength and durability (Borden Bubble Sci Eng Technol. 2009 November; 1(1-2): 3-17). Encapsulated cells, biologicals and macromolecules have been used in many therapeutic and non-therapeutic application.

Molecular methods and assays often use micro-bubbles as compartments to hold nanoliter (nL) volumes for molecular analysis. It is well known that individual nucleic acids can be captured into a nL sized compartment. For example, the micro-bubbles or “droplets” can serve as compartments for molecular reactions such as polymerase chain reaction (PCR) amplification. This the principle used in digital droplet PCR methods (WO 2010018465 A2, U.S. Pat. No. 8,535,889 B2) where a single copy of a gene is captured into the nL sized droplet and amplified by PCR with a fluorescence signal generated in a droplet when the gene is present.

Current approaches need to minimized the number of compartments to 10⁵ or less due to the time it takes to read and/or sort droplets. This means that droplets are likely to be contaminated with interfering or redundant molecules unless many droplets are empty (typically 99%) and allow only one molecule per droplet. The large number of empty compartments reduced the detection limit of the digital droplet PCR measurement and makes measurement of vet low abundancy (0.1%) molecules difficult in droplets.

However any artificial cells application need to generate large libraries of droplet compartments. Often as many as up to 10⁹ and look for exceeding rare abundancy (1 in a million) of desired molecules (Guo Lab on a chip 2012 DOI: 10.1039/c21c21147e). For example, when generating clone libraries housed in micro bubbles there might be millions or billions of genetic alterations possible and only a handful of mutants with desired properties. Therefore there is an unmeet need for rapid analysis up to 10⁹ compartments or droplets.

Most digital droplet analysis is based on a microfluidic capillary to sort and read individual each droplet as either full or empty (e.g. Larson US2011/0244455). This approach makes use of flowing droplets through microfluidic channel while simultaneously passing through detector. The problem with this “microfluidic cytometry” approach is that the empty drop must be read and this slows throughput, thus the system is still limited to 10⁵ droplets/hour. Droplet generation is also still limited to 10⁵ droplets/hour and the method cannot avoid contamination issue and must generate excess empty droplets and keep the abundance of full drops low.

Meanwhile the generation of droplets by electrospray can produce droplets at a rate of 10⁶ per second (Chang EPL (Europhysics Letters) 99.6 (2012): 64003). These droplets can have diameters in the range of 1 μm to 200 μm. However, while it is possible to generate 10⁹ droplets in minutes by electrospray , the issue remains how to analyze the droplets as fast. The common approach used in “microfluidic cytometry” is to sort the droplets to enrich the full droplets. These approaches use a capillary with a Y or T junction (Whitesides Lab Chip, 2006, 6, 437-446) and a capacitance sensor (Sohn PNAS 2000,97,20) which can detect the full droplets and deflect droplets to a desired location. While this method can enrich droplets, the key problem still remains that each droplet must be read and the slows throughput of the system remains and the methods still are limited to analytics of 10⁵ or less droplets in hours. Microfluidic cytometry is similar in speed and throughput demonstrated with fluorescence-activated cell sorting (FACS) systems which can also be used for droplets sorting (Nucleic Acid Quantitation in Cells Fixed for FACS PLoS ONE 8(9):e73849).

Several alternative approaches are known for sorting cells rapidly. Magnetic beads bound to cells are commonly used for sorting cells (U.S. Pat. No. 5,968,820; 1999). Membrane size exclusion has long been used for sorting cells by filtration (Seal S H, Cancer 1964 17, 637-42, WO2005/047529). Here the pore diameters on the porous material are kept small enough to retain larger sized cells. Both method have the benefit from washing away unbound material. Separation of droplets by filtration is possible with larger droplets retained on membrane surfaces (Varanasi. Scientific reports 2014;4:5504). However, none of these approaches are able to perform reliable droplet sorting as emulsion stability can be an issue with sedimentation, aggregation, coalescence and phase inversion occurring. The oil polarity, temperature, nature of solids in the droplet, droplet size and pH all can impact emulsion stability. This leads to loss of key droplets and contents.

While encapsulation of cells, biologicals and macromolecules by micro bubbles or droplets allows many therapeutic and non-therapeutic applications, current approaches minimized the number of droplets to 10⁵ or less due to the time it takes to read and/or sort droplets. This means that droplets are likely to be contaminated with interfering or redundant molecules when >1% are full. Another issue is that a larger number of droplets are empty (>99.9%) when the low abundancy (0.1%) molecules are measured in samples. Therefore, there is a long felt need for rapid analysis up to 10⁹ compartments or droplets to look for exceedingly rare abundancy (1 in a million) of desired molecules

### SUMMARY OF THE INVENTION

The invention is a means of allowing generation of higher numbers of droplets containing content while still diluting the interfering molecules to allow generating large arrays of isolated droplets without increased need to sort more droplets while allowing a higher content. The invention generates a larger group of droplets containing a library of compounds, then removes the empty droplets and retain contents of full droplets by size exclusion filtration.

The key features of this invention are: (1) generation of a set of droplets (10⁶ or greater) for encapsulation of a compound library (10² or greater) such that ratio of full to empty droplet allows of dilution of sample interference (dilution of 3:100 or greater); (2) removal of empty droplets by destabilized of emulsion so contents are spilled into oil phase and; (3) capture of full droplets or their content on to a porous matrix by size exclusion filtration.

This invention works with droplets which are retained by exclusion filtration and can include the following features: (1) a droplet containing a compound of any combination of one or more cells, particles, biologicals, macromolecules or; (2) droplets which are produced rapidly more 10³/sec; (3) a stabilization agent added to the either immiscible liquid; (4) a destabilization agent added to either immiscible liquid; (5) instances where droplets are filtered onto a porous matrix; (6) instances where full droplets are destabilized after empty droplets are removed; (7) instances where droplet and particle size can be varied from 1 to 200 μm to improve separation; and (8) instances where pore size and shape can be varied to improve isolation of empty droplets from retained content.

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIG. 1 is a schematic depicting an example of a method in accordance with the invention described herein for droplet generation. As shown in FIG. 1, a cell or particle in a biological solution 1 is pushed through a capillary into an immiscible liquid 2 with or without an emulsifier to generate 3 the droplets such that approximately 10⁸ droplets 4 or more are made with a small fraction, for example 0.1%, being filled with single cell or single particle. The empty droplets are removed 5 leaving approximately 10⁵ full droplets.

FIG. 2 is another schematic depicting an example of a method in accordance with the invention described herein for empty droplet removal where cells or particles in droplets 6 are loaded on to porous matrix 7 for size exclusion filtration and droplet stabilizing liquid 8 is removed through pores of the porous matrix, a droplet breaking liquid 9 is added and empty droplets 10 are removed. The retained droplets having molecules, cells, and particles 11 are captured in a porous matrix. The contents 12 of are then removed through pores of the porous matrix.

FIGS. 3 and 4 are the same schematics of FIGS. 1 and 2 explaining with words in more details the method of the invention.

### DETAILED DESCRIPTION OF THE INVENTION

Methods, apparatus and kits in accordance with the invention described herein have application in any situation where detection or isolation of rare molecules and cells is needed. Examples of such applications include, by way of illustration and not limitation, diagnostics, biological reactions, chemical reactions, high through-put screening, cloning, clone generation, artificial cells, regenerative cells, compound libraries, cell library screening, cell culturing, protein engineering and other applications.

Some examples in accordance with the invention described herein are directed to methods of molecular analysis. Other examples in accordance with the principles described herein are directed to methods of isolation, characterization and detection of cells, particles, macromolecules, genes, proteins, biochemicals, organic molecules or other compounds. While other examples use droplet sorting for detection of rare cells and cell free molecules. Other examples in accordance with the principles described herein are directed to methods of selective detection of genes, proteins, cells and biomarkers.

Other examples in accordance with the invention described herein are directed to methods of binding and separation of cells and cellular biological content whereby cells are isolated on a porous matrix and bound materials retained for analysis. In some cases the cells are artificial cells, modified cells, natural cells, of any and all types.

Some examples in accordance with the invention described herein are directed to methods of binding and separation of nucleic acid, proteins or other biological molecules on to where particles are isolated on porous matrix or by magnetic particle and bound materials retained for analysis.

Some examples in accordance with the invention described herein are directed to methods of detecting one or more different populations of nucleic acid, proteins or other biological molecules rare molecules in a sample suspected of containing the one or more different populations of rare molecules and non-rare molecules. These nucleic acid, proteins or other biological molecules can be used as ligand binding measures of cells, enzymes, proteases, receptors, proteins, nucleic acid, peptidase, proteins, inhibitors and the like by acting on formation or binding of said molecules. These molecules can be formed as metabolites, natural or man-made origin, such as biological, therapeutics, or others.

Examples in accordance with the principles described herein are directed to methods and kits for molecular, protein or biological molecule analysis. Other examples in accordance with the invention described herein are directed to apparatus for analysis.

Common terminology used to describe this invention are “droplet”, “compounds” “in excess”, “rapid”, “emulsion”, “size exclusion filtration”, “compound library”, and are defined further below.

A “droplet” is a micro-bubble defined as a compartment to hold nanoliter (nL) volumes of biological fluidics and compounds. The droplet can contain compounds and be considered “full”. The droplet can lack compounds and be considered “empty”. The “compounds” can be cells, particles, macromolecules, genes, proteins, biochemicals, organic molecules, or others. The droplet size can be varied to reduce the space allowed for a compound, for example the droplet can be nm to μm in diameter. An “excess” of empty droplets to full droplets means a ratio of no greater than 10 full droplets:100 empty droplets such that the ratio of empty to full droplet allows dilution of sample interference. “Rapid” droplet generation and sorting means at least>10²/sec.

An “emulsion” is created when the droplet separate in two immiscible liquids, namely a generally “aqueous phase” held inside the droplet and a generally “oil phase” outside the droplet. Emulsifiers, surfactants, polar, apolar solvents, solutes and the droplets are considered components of an “emulsion”. The stabilization or destabilization of an “emulsion” can lead to continuation of the “emulsion” or separation of aqueous and oil into separate phases without “droplet”

“Size exclusion filtration” is the use of a porous matrix to separate droplets and the contents from the rest of the emulsion. The contents of the droplets are retained on the porous matrix and are called “retained contents”. “Retained contents” can be cells or particles and associated molecules. Pore diameters of the porous matrix are kept small enough to retain larger sized droplets and their contents. “Size exclusion filtration” allow washing away unbound material or material not in full droplets or associated with retained contents.

A “library of compounds” is a group including organic molecules, biochemical, genes, particulates, cells, or macromolecules which contain unique group members. Generally, the library is a group of compounds of similar size and nature and contains some molecule differences between group members. A library of compounds can be a group “variations of peptides and proteins” or variations of nucleic acids such as sequence differences. The “library of compounds” can be captured onto “capture particles”, macromolecules or cells. The “library of compounds” can be captured through an “affinity agent”. Encapsulation of a compound library in a droplet is typically at at least 10² different group members.

The term “variations of peptides and proteins” is a part, piece, fragment or modification of a “polypeptide,” “peptide” and protein of biological or non-biological origin.

The term “label particle” refers to a particle bound to mass label agent. This particle can additional be bound to affinity agent or affinity tags.

The term “capture particle” refers to a particle attached to an affinity agent

The term “affinity agent” refers to a molecule capable of selectively binding to a specific molecule. The affinity agent can direct bind the rare molecule of interest, the mass label or an affinity tag. Affinity agent can be attached to a capture particle or label particles or can bind a particle through the affinity for the mass label, rare molecule or affinity tag on label particle

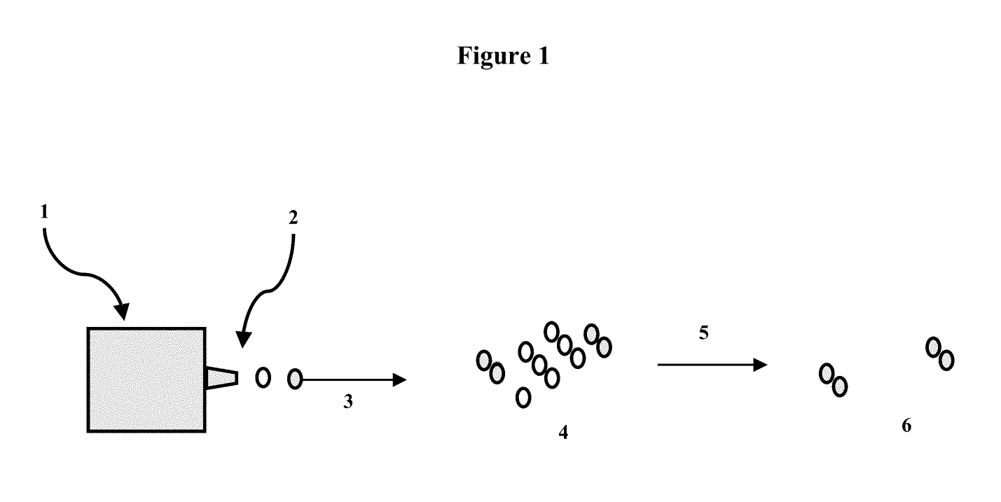

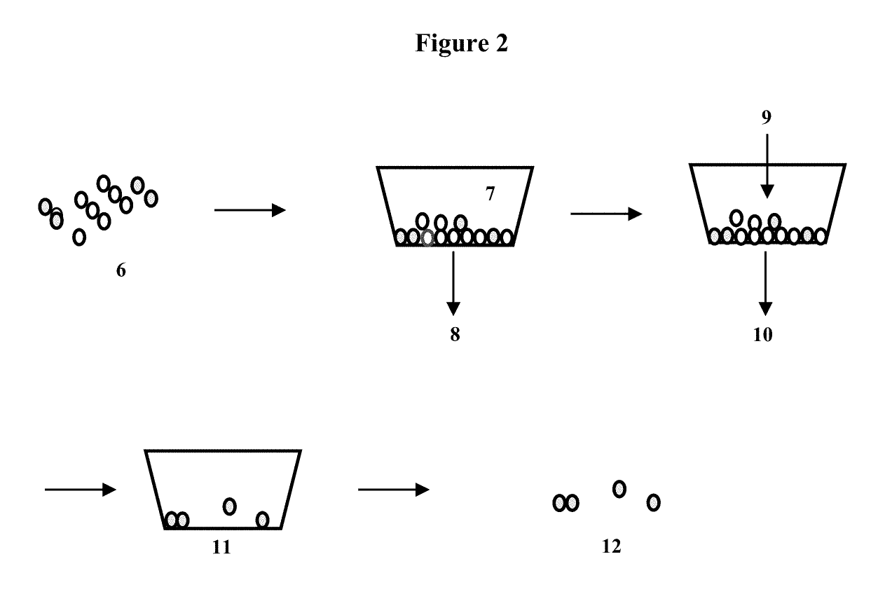

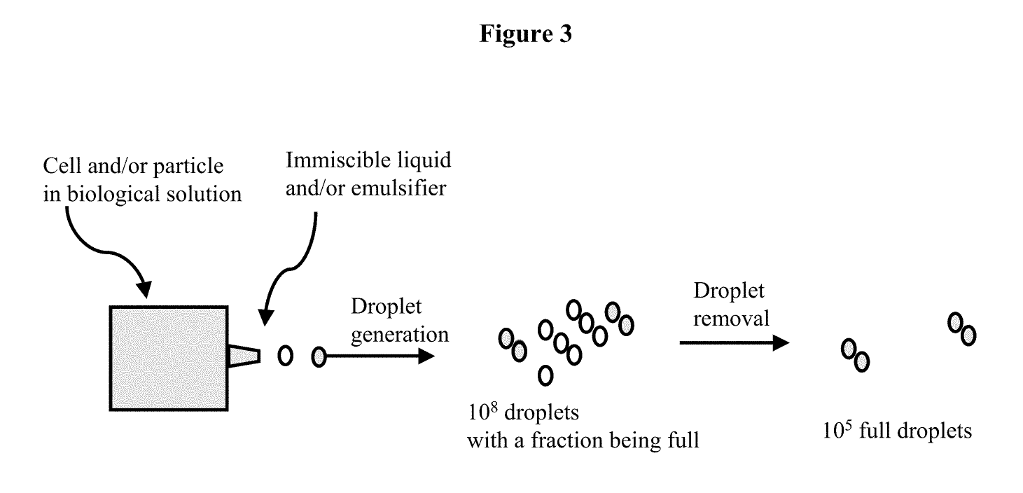

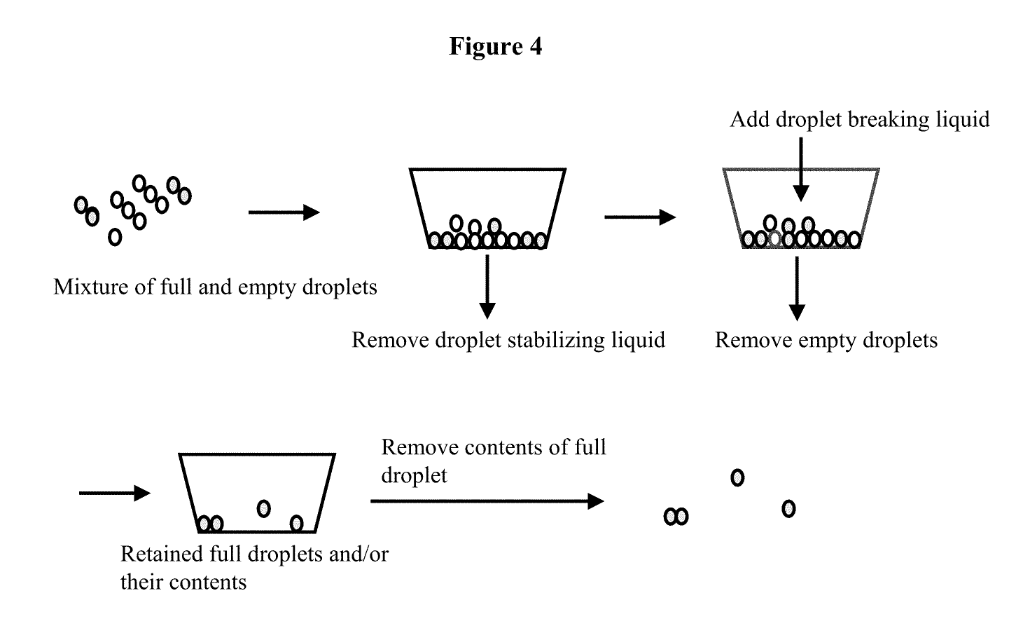

An example of a method for detection of rare molecules in accordance with the principles described herein is depicted in FIGS. 1 and 2 as described above in description of the figures and is an example of generating the droplets containing a library of compounds in an emulsion and removing the empty droplets but retaining contents of full droplets by size exclusion filtration. The size exclusion filtration allows the oil phase to pass through porous matrix.

In some examples, empty droplets are destabilized into oil phase. The content released form the empty droplets and passes through a porous matrix. In other examples, full droplets are stabilized to not spill into the oil phase. The retained contents are not released from droplets and do not passes through porous matrix. In still other examples, the full and empty droplets are destabilized into oil phase. The content released from the empty droplets and passes through porous matrix. The retained contents are released form full droplets but do not pass through the porous matrix.

In some examples the retained content can be a retained on porous matrix in liquid holders. The retained contents can be on a particle isolated on porous matrix. The retained contents can be a cell isolated on a porous matrix. The retained contents can be a droplet isolated on a porous matrix. The retained contents can be a molecule isolated on porous matrix.

### Examples of Variations of Droplets

A droplet is a micro-bubbles defined as a compartment to hold nanoliter (nL)) to microliter (μL) volume of biological fluidics and compounds. The compounds can be organic molecules, biochemical, particles, cells, or other macromolecules. The biological fluidics are aqueous or polar solutions that can contain solutes, polymers, surfactants, emulsifiers, macromolecules, other solvents, and particles in addition to the compounds. The droplet can contain compounds and be considered full. The droplet can lack compounds and be considered empty. The droplet size can be varied to reduce the space allowed for a compound. The droplet size can be varied reduce the space allowed for a compound, for example the droplet can be varied from 1 to 400 μm diameter that hold nL to μL volumes.

The number of empty droplets compared to the number of full droplets can be large (>97%) with small only (<3%) of droplets created full. In some examples the ratio of full to empty droplets is about 1 to 100, or about 1 to 1000, or about 1 to 10000.

The droplets are made when an emulsion is created causing the separation of two immiscible liquids, “aqueous phase” held inside the droplet and a generally “oil phase” outside the droplet. Aqueous phases can include hydrophilic chemical and biochemicals such as water, polar protic solvents, polar aprotic solvent and mixtures thereof. Oil phase can include organic solvents, oils such as vegetable, synthetics, animal products, lipids and other lipophilic chemicals and biochemicals. The emulsion can be oil-in-water, water in oil, water in oil in water, and oil in water in oil Emulsifiers, emulgents, surfactants can be considered components of the emulsion to change the surface energy of the droplet or the hydrophilic/hydrophobic (lipophilic) balance and include anionic, cationic, nonionic and amphoteric surfactants, as well as naturally occurring materials. Emulsion instability can cause sedimentation, aggregation, coalescence and phase inversion. The emulsion stability can be impacted by oil polarity, temperature, nature of solids in the droplet, droplet size and pH. These properties can be used to stabilize or destabilize droplets and contents.

### Examples of Variations of Peptides and Proteins

In accordance with the invention described herein, a “variations of peptides and proteins” can be derived from a peptide or protein from biological or non-biological origin. The variations of peptides and proteins can be used to measure diseases. The variations of peptides and proteins can be as the result of disease or intentional reactions. The variations of peptides and proteins can result in proteins and peptides of man-made or natural origin and include bioactive and non-bioactive peptide or protein such as those used in medical devices, therapeutic use, for diagnostic use, used for measurement of processes, and those used as food, in agriculture, in production, as pro or prebiotics, in micro-organism or cellular production, as chemicals for processes, for growth, measurement or control of cells, used for food safety and environmental assessment, used in veterinary products, and used in cosmetics. The fragments can be used to measure enzymes and peptidase of interest based on formation of variations of peptides and proteins. The variations of peptides and proteins can be used to measure natural or synthetic inhibition of enzymes and peptidase inhibitors of interest based on lack formation of fragments.

The variations of peptides and proteins can be as the result of translation, or posttranslational modification by enzymatic or non-enzymatic modifications. Post-translational modification refers to the covalent modification of proteins during or after protein biosynthesis. Post-translational modification can be through enzymatic or non-enzymatic chemical reaction. Phosphorylation is a very common mechanism for regulating the activity of enzymes and is the most common post-translational modification. Enzymes can be oxidoreductases, hydrolases, lyases, isomerases, ligases or transferases as known commonly in enzyme taxonomy databases, such as http://enzyme.expasy.org/ or http://www.enzyme-database.org/ which have more than 6000 entries.

Common modification of variations of peptides and proteins include the addition of hydrophobic groups for membrane localization, addition of cofactors for enhanced enzymatic activity, diphthalamide formation, hypusine formation, ethanolamine phosphoglycerol attachment, diphthalamide formation, acylation, alkylation, amide bond formation such as amino acid addition or amidation, butyrylation gamma-carboxylation dependent on Vitamin K[15], glycosylation, the addition of a glycosyl group to either arginine, asparagine, cysteine, hydroxylysine, serine, threonine, tyrosine, or tryptophan resulting in a glycoprotein., malonylationhydroxylation, iodination, nucleotide addition such as ADP-ribosylation, phosphate ester (O-linked) or phosphoramidate (N-linked) formation such as phosphorylation or adenylylation, propionylation pyroglutamate formation, S-glutathionylation, S-nitrosylation S-sulfenylation (aka S-sulphenylation, succinylation or sulfation). Nonenzymatic modification include the attachment of sugars, carbamylation, carbonylation or intentional recombinate or synthetic conjugation such as biotinylation or addition affinity tags, like His oxidation, formation of disulfide bonds between Cys residues or pegylation.

Common reagents for intentional fragmentation to variations of peptides and proteins include peptidases or reagents known to react with peptides and proteins. Intentional fragmentation can generate specific fragments and uses predicted cleavage sites for proteases (also termed peptidases or proteinases) and chemicals known to react with peptide and protein sequence. Common peptidases and chemicals for intentional fragmentation include Arg-C, Asp-N, BNPS oNCS/urea, caspase, chymotrypsin (low specificity), Clostripain, CNBr, enterokinase, factor Xa, formic acid, Glu-C, granzyme B, HRV3C protease, hydroxylamine, iodosobenzoic acid, Lys-C, Lys-N, Mild acid hydrolysis, NBS, NTCB, elastase, pepsin A, prolyl endopeptidase, proteinase K, TEV protease, thermolysin, thrombin, and trypsin. Common reagents for intentional inhibition of fragmentation include peptidase and chemical inhibitors for peptidases and chemicals above listed.

### Examples of Affinity Agent

An affinity agent is a molecule capable of binding selectively to a rare molecule or mass labels. Selective binding involves the specific recognition of one of two different molecules for the other compared to substantially less recognition of other molecules. The terms “binding” or “bound” refers to the manner in which two moieties are in association to one another.

An affinity agent is a molecule capable of binding selectively to a rare molecule or mass labels. Selective binding involves the specific recognition of one of two different molecules from the other compared to substantially less recognition of other molecules. The terms “binding” or “bound” refers to the manner in which two moieties are associated to one another. An affinity agent can be a immunoglobulin, protein, peptide, metal, carbohydrate, metal chelator, nucleic acid or other molecule capable of binding selectively to a particular rare molecule or a mass labels type.

Examples of nucleic acids including but not limited include natural or made-made oligomeric nucleic acids. The oligomeric nucleic acid may be any polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, silencing (siRNA), xeno nucleic acids (XNA), recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer.

The sequence of nucleotides may be interrupted by non-nucleotide components. A polynucleotide may be further modified, such as by conjugation with a labeling component. The terms “isolated nucleic acid” and “isolated polynucleotide” are used interchangeably; a nucleic acid or polynucleotide is considered “isolated” if it: (1) is not associated with all or a portion of a polynucleotide in which the “isolated polynucleotide” is found in nature, (2) is linked to a polynucleotide to which it is not linked in nature, or (3) does not occur in nature as part of a larger sequence.

The affinity agents which are immunoglobulins may include complete antibodies or fragments thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, and Fab′, for example. In addition, aggregates, polymers, and conjugates of immunoglobulins or their fragments can be used where appropriate so long as binding affinity for a particular molecule is maintained. Antibodies can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies.

Polyclonal antibodies and monoclonal antibodies may be prepared by techniques that are well known in the art. For example, in one approach monoclonal antibodies are obtained by somatic cell hybridization techniques. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In general, monoclonal antibodies can be purified by known techniques such as, but not limited to, chromatography, e.g., DEAE chromatography, ABx chromatography, and HPLC chromatography; and filtration, for example.

An affinity agent can additionally be a “cell affinity agent” capable of binding selectively to a rare molecule which is used for typing a rare cell or measuring a biological intracellular process of a cell. These rare cell markers can be immunoglobulins that specifically recognizes and binds to an antigen associated with a particular cell type and whereby antigen are components of the cell. The cell affinity agent is capable of being absorbed into or onto the cell.

The term “cell affinity agent” refers to a rare cell typing markers capable of binding selectively to rare cell. Selective cell binding typically involves “binding between molecules that is relatively dependent of specific structures of binding pair. Selective binding does not rely on non-specific recognition.

### Examples of Label and Capture Particles

Affinity agent can be attached to mass labels and/or particles for purpose of detection or isolation of rare molecules. This attachment can occur through “label particles” which are in turn attached to mass labels. Affinity agents can also be attached to “capture particles” which allow separation of bound and unbound mass labels or rare molecule. This attachment to capture and label can be prepared by directly attaching the affinity agent onto a “linking group”. The terms “attached” or “attachment” refers to the manner in which two moieties are connected by a direct bond between the two moieties or a linking group between the two moieties. This allows the method to be multiplexed for more than one result at a time. Alternatively, affinity agent can be attached to mass labels and/or particles mass label using additional “binding partners”. The phrase “binding partner” refers to a molecule that is a member of a specific binding pair of affinity agent and “affinity tags” that bind each other and not the mass labels or rare molecules. In some cases, the affinity agent may be members of an immunological pair such as an antigen to antibody or hapten to antibody, biotin to avidin, IgG to protein A, secondary antibody to primary antibody, antibodies to fluorescent labels and other binding pairs.

The “label particle” is a particulate material which can be attached to the affinity agent through a direct linker arm or a binding pair. Also the “labeled particle” is capable of forming an X-Y cleavable linkage between labeled particle and mass label. The size of the labeled particle is large enough to accommodate one or more mass labels and affinity agents. The ratio of affinity agents or mass label to a single label particle may be 10⁷ to 1, 10⁶ to 1, or 10⁵ to 1, or 10⁴ to 1, or 10³ to 1, or 10² to 1, or 10 to 1, for example. The number of affinity agents and mass labels associated with the label particle is dependent on one or more of the nature and size of the affinity agent, the nature and size of the label particle, the nature of the linker arm, the number and type of functional groups on the label particle, and the number and type of functional groups on the mass label, for example.

The composition of the label or capture particle entity may be organic or inorganic, magnetic or non-magnetic as a nanoparticle or a micro particle. Organic polymers include, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinylbenzene), poly(styrene/acrylate), poly(ethylene terephthalate), demdrimer, melamine resin, nylon, poly(vinyl butyrate), for example, either used by themselves or in conjunction with other materials and including latex, microparticle and nanoparticle forms thereof. The particles may also comprise carbon (e.g., carbon nanotubes), metal (e.g., gold, silver, and iron, including metal oxides thereof), colloids, dendrimers, dendrons, and liposomes, for example. In some examples, the label particle may be a silica nanoparticle. In other some examples, labeled particles can be magnetic that have free carboxylic acid, amine or tosyl groups. In other some examples, label particles can be mesoporous and include mass labels inside the label particles.

The diameter of the label or capture particle is dependent on one or more of the nature of the rare molecule, the nature of the sample, the permeability of the cell, the size of the cell, the size of the nucleic acid, the size of the affinity agent, the magnetic forces applied for separation, the nature and the pore size of a filtration matrix, the adhesion of the particle to matrix, the surface of the particle, the surface of the matrix, the liquid ionic strength, liquid surface tension and components in the liquid, and the number, size, shape and molecular structure of associated label particles, for example.

The term “permeability” means the ability of a particles and molecule to enter a cell through the cell wall. In the case of detection of a rare molecule inside the cell, the diameter of the labeled particles must be small enough to allow the affinity agents to enter the cell. The label particle maybe coated with materials to increase “permeability” like collagenase, peptides, proteins, lipid, surfactants, and other chemicals known to increase particle inclusion into the cell.

When a porous matrix is employed in a filtration separation step, the diameter of the labeled particles must be small enough to be pass through the pores of a porous matrix if it did bind the rare molecule, and the diameter of the label particles must be large enough to not pass through the pores of a porous matrix to retain the bound rare molecule on the matrix. In some examples in accordance with the invention described herein, the average diameter of the label particles should be at least about 0.01 microns (10 nm) and not more than about 10 microns In some examples, the particles have an average diameter from about about 0.02 microns to about 0.06 microns, or about 0.03 microns to about 0.1 microns, or about 0.06 microns to about 0.2 microns, or about 0.2 microns to about 1 micron, or about 1 micron to about 3 microns, or about 3 micron to about 10 microns. In some examples, the adhesion of the particles to the surface is so strong that the particle diameter can be smaller than the pore size of the matrix.

The affinity agent can be prepared by directly attaching the affinity agent to a carrier or capture particles by linking groups. The linking group between the labeled particle and the affinity agent, may be aliphatic or aromatic bond. The linking groups may comprise a cleavable or non-cleavable linking moiety. Cleavage of the cleavable moiety can be achieved by electrochemical reduction used for the mass label but also may be achieved by chemical or physical methods, involving furthers oxidation, reduction, solvolysis, e.g., hydrolysis, photolysis, thermolysis, electrolysis, sonication, and chemical substitution, for example. Photocleavable bonds that are cleavable with light having an appropriate wavelength such as, e.g., UV light at 300 nm or greater; for example. The nature of the cleavage agent is dependent on the nature of the cleavable moiety. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers, ureas, thioureas, guanidines, azo groups, thioethers, carboxylate and so forth. The linking group may also be a macro-molecule such as polysaccharides, peptides, proteins, nucleotides, and dendrimers.

The linking group between the particle and the affinity agent may be a chain of from 1 to about 60 or more atoms, or from 1 to about 50 atoms, or from 1 to about 40 atoms, or from 1 to 30 atoms, or from about 1 to about 20 atoms, or from about 1 to about 10 atoms, each independently selected from the group normally consisting of carbon, oxygen, sulfur, nitrogen, and phosphorous, usually carbon and oxygen. The number of heteroatoms in the linking group may range from about 0 to about 8, from about 1 to about 6, or about 2 to about 4. The atoms of the linking group may be substituted with atoms other than hydrogen such as, for example, one or more of carbon, oxygen and nitrogen in the form of, e.g., alkyl, aryl, aralkyl, hydroxyl, alkoxy, aryloxy, or aralkoxy groups. As a general rule, the length of a particular linking group can be selected arbitrarily to provide for convenience of synthesis with the proviso that there is minimal interference caused by the linking group with the ability of the linked molecules to perform their function related to the methods disclosed herein.

Obtaining reproducibility in amounts of particle captured after separation and isolation is important for rare molecular analysis. Additionally, knowing the amounts of particle captured that enter a rare cell is important to maximize the amount of specific binding. Knowing the amount of particles remaining after washing are important to minimize the amount of non-selective binding. In order to make these determination, it is helpful if the particles can contain fluorescent, optical or chemiluminescence labels. Therefore, labeled particles, can be measured by fluorescent or chemiluminescence by virtue of the presence of a fluorescent or chemiluminescence molecule. The fluorescent and optical molecule can then be measured by microscopic analysis and compared to expected results for sample containing and lacking analyte. Fluorescent molecules include but not limited to dylight™, FITC, rhodamine compounds, phycoerythrin, phycocyanin, allophycocyanin, o-phthalaldehyde, fluorescent rare earth chelates, amino-coumarins, umbelliferones, oxazines, Texas red, acridones, perylenes, indacines such as, e.g., 4,4-difluoro-4-bora-3a,4a-diaza-s-indacene and variants thereof, 9,10-bis-phenylethynylanthracene, squarine dyes and fluorescamine, for example. A fluorescent microscope or fluorescent spectrometer may then be used to determine the location and amount of the labeled particles. Chemiluminescence labels examples include luminol, acridinium esters and acridinium sulfonamides to name a few. Optical labels examples include color particles, gold particles and enzymatic colorimetric reactions to name a few.

### Examples of Porous Matrix and Filtration

Porous matrices are used in “size exclusion filtration” to allow washing away unbound material or material not in full droplets or associated with retained contents. The contents of the droplets are retained on the porous matrix and are called “retained contents”. “Retained contents” can be cells or particles and associated molecules. Full droplets also can be retained with contents on the porous matrix. Pore diameters of the porous matrix are kept small enough to retain larger sized droplets and their contents. “Size exclusion filtration” allows washing away unbound material or material not in full droplets or associated with retained contents.

In some methods in accordance with the principles described herein, the sample is incubated with an affinity agent comprised of a mass label and label particle, for each different population of rare molecules. The affinity agent that comprises a specific binding partner that is specific for and binds to a rare molecule of one of the populations of the rare molecules. The rare molecules can be cell bound or cell free. The affinity agent with mass label and labeled particle are retained on the surface of a membrane after a filtration.

The separation can occur in some examples when the porous matrix employed in the filtration separation step is such that the pore diameter is smaller than the diameter of the cell with the rare molecule but larger that the unbound labeled particles to allow the affinity agents to achieve the benefits of rare molecule capture in accordance with the principles described herein but small enough to pass through the pores of a porous matrix if it did not capture rare molecules. In other methods, the porous matrix employed in the filtration separation step is such that the pore diameter is smaller than the diameter of the affinity agents on the labeled particle capable of binding rare molecule but larger that the unbound molecule pass through allowing the affinity agents to achieve the benefits of rare molecule capture. In still other methods, the affinity agents on labeled particles can be additionally bound through “binding partners” or sandwich assays of other capture particles, like magnetic particles, or to a surface, like a membrane. In the later case, the capture particles are retained on the surface of the porous membranes.

In all examples, the concentration of the one or more different populations of rare molecules is enhanced over that of the non-rare molecules to form a concentrated sample. In some examples, the sample is subjected to a filtration procedure using a porous matrix that retains the rare molecules while allowing the non-rare molecules to pass through the porous matrix thereby enhancing the concentration of the rare molecules. In the event that one or more rare molecules are non-cellular, i.e., not associated with a cell or other biological particle, the sample is combined with one or more capture particle entities wherein each capture particle entity comprises a binding partner for the non-cellular rare molecule of each of the populations of non-cellular rare molecules to render the non-cellular rare molecules in particulate form, i.e., to form particle-bound non-cellular rare molecules. The combination of the sample and the capture particle entities are held for a period of time and at a temperature to permit the binding of non-cellular rare molecules with corresponding binding partners of the capture particle entities. Vacuum is then applied to the sample on the porous matrix to facilitate passage of non-rare cells and other particles through the matrix. The level of vacuum applied is dependent on one or more of the nature and size of the different populations of rare cells and/or particle reagents, the nature of the porous matrix, and the size of the pores of the porous matrix, for example.

Contact of the sample with the porous matrix is continued for a period of time sufficient to achieve retention of cellular rare molecules and/or particle-bound non-cellular rare molecules on a surface of the porous matrix to obtain a surface of the porous matrix having different populations of rare cells and/or particle-bound rare molecules as discussed above. The period of time is dependent on one or more of the nature and size of the different populations of rare cells and/or particle-bound rare molecules, the nature of the porous matrix, the size of the pores of the porous matrix, the level of vacuum applied to the blood sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

An amount of each different affinity agent that is employed in the methods in accordance with the principles described herein is dependent on one or more of the nature and potential amount of each different population of rare molecules, the nature of the mass label, the natured of attachment, the nature of the affinity agent, the nature of a cell if present, the nature of a particle if employed, and the amount and nature of a blocking agent if employed, for example. In some examples, the amount of each different modified affinity agent employed is about 0.001 μg/μL to about 100 μg/μL, or about 0.001 μg/μL to about 80 μg/μL, or about 0.001 μg/μL to about 60 μg/μL, or about 0.001 μg/μL to about 40 μg/μL, or about 0.001 μg/μL to about 20 μg/μL, or about 0.001 μg/μL to about 10 μg/μL, or about 0.5 μg/μL to about 100 g/μL, or about 0.5 μg/μL to about 80 μg/μL, or about 0.5 μg/μL, to about 60 μg/μL, or about 0.5 μg/μL to about 40 μg/μL, or about 0.5 μg/μL to about 20 μg/μL, or about 0.5 μg/μL to about 10 μg/μL, for example.

The porous matrix is a solid, material, which is impermeable to liquid (except through one or more pores of the matrix in accordance with the principles described herein. The porous matrix is associated with a porous matrix holder and plastic like a ELISA plate or a vail. The association between porous matrix and holder can be done with an adhesive. The association between porous matrix in the holder and the liquid holder can be through direct contact or with a flexible gasket surface.

The porous matrix is a solid or semi-solid material and may be comprised of an organic or inorganic, water insoluble material. The porous matrix is non-bibulous, which means that the membrane is incapable of absorbing liquid. In some examples, the amount of liquid absorbed by the porous matrix is less than about 2% (by volume), or less than about 1%, or less than about 0.5%, or less than about 0.1%, or less than about 0.01%, or 0%. The porous matrix is non-fibrous, which means that the membrane is at least 95% free of fibers, or at least 99% free of fibers, or at least 99.5%, or at least 99.9% free of fibers, or 100% free of fibers.

The porous matrix can have any of a number of shapes such as, for example, track-etched, or planar or flat surface (e.g., strip, disk, film, matrix, and plate). The matrix may be fabricated from a wide variety of materials, which may be naturally occurring or synthetic, polymeric or non-polymeric. The shape of the porous matrix is dependent on one or more of the nature or shape of holder for the membrane, of the microfluidic surface, of the liquid holder, of cover surface, for example. In some examples the shape of the porous matrix is circular, oval, rectangular, square, track-etched, planar or flat surface (e.g., strip, disk, film, membrane, and plate), for example.

The porous matrix and holder may be fabricated from a wide variety of materials, which may be naturally occurring or synthetic, polymeric or non-polymeric. Examples, by way of illustration and not limitation, of such materials for fabricating a porous matrix include plastics such as, for example, polycarbonate, poly (vinyl chloride), polyacrylamide, polyacrylate, poly-ethylene, polypropylene, poly(4-methylbutene), polystyrene, polymethacrylate, poly(ethylene terephthalate), nylon, poly(vinyl butyrate), poly(chlorotrifluoroethylene), poly(vinyl butyrate), polyimide, polyurethane, and parylene, silanes, silicon, silicon nitride, graphite, ceramic material (such, e.g., as alumina, zirconia, PZT, silicon carbide, aluminum nitride); metallic material (such as, e.g., gold, tantalum, tungsten, platinum, and aluminum); glass (such as, e.g., borosilicate, soda lime glass, and PYREX®); and bioresorbable polymers (such as, e.g., poly-lactic acid, polycaprolactone and polyglycolic acid); for example, either used by themselves or in conjunction with one another and/or with other materials. The material for fabrication of the porous matrix and holder are non-bibulous does not include fibrous materials such as cellulose (including paper), nitrocellulose, cellulose acetate, rayon, diacetate, lingins, mineral fibers, fibrous proteins, collagens, synthetic fibers (such as nylons, dacron, olefin, acrylic, polyester fibers, for example) or, other fibrous materials (glass fiber, metallic fibers), which are bibulous and/or permeable and, thus, are not in accordance with the principles described herein. The material for fabrication of the porous matrix and holder may be the same or different materials.

The porous matrix for each liquid holder comprises at least one pore and no more than about 2,000,000 pores per square centimeter (cm²). In some examples, the number of pores of the porous matrix per cm² is 1 to about 2,000,000, or 1 to about 1,000,000, or 1 to about 500,000, or 1 to about 200,000, or 1 to about 100,000, or 1 to about 50,000, or 1 to about 25,000, or 1 to about 10,000, or 1 to about 5,000, or 1 to about 1,000, or 1 to about 500, or 1 to about 200, or 1 to about 100, or 1 to about 50, or 1 to about 20, or 1 to about 10, or 2 to about 500,000, or 2 to about 200,000, or 2 to about 100,000, or 2 to about 50,000, or 2 to about 25,000, or 2 to about 10,000, or 2 to about 5,000, or 2 to about 1,000, or 2 to about 500, or 2 to about 200, or 2 to about 100, or 2 to about 50, or 2 to about 20, or 2 to about 10, or 5 to about 200,000, or 5 to about 100,000, or 5 to about 50,000, or 5 to about 25,000, or 5 to about 10,000, or 5 to about 5,000, or 5 to about 1,000, or 5 to about 500, or 5 to about 200, or 5 to about 100, or 5 to about 50, or 5 to about 20, or 5 to about 10, for example. The density of pores in the porous matrix is about 1% to about 20%, or about 1% to about 10%, or about 1% to about 5%, or about 5% to about 20%, or about 5% to about 10%, for example, of the surface area of the porous matrix. In some examples, the size of the pores of a porous matrix is that which is sufficient to preferentially retain liquid while allowing the passage of liquid droplets formed in accordance with the principles described herein. The size of the pores of the porous matrix is dependent on the nature of the liquid, the size of the cell, the size of the capture particle, the size of mass label, the size of an analyte, the size of label particles, the size of non-rare molecules, and the size of non-rare cells, for example. In some examples, the average size of the pores of the porous matrices is about 0.1 to about 20 microns, or about 0.1 to about 5 microns, or about 0.1 to about 1 micron, or about 1 to about 20 microns, or about 1 to about 5 microns, or about 1 to about 2 microns, or about 5 to about 20 microns, or about 5 to about 10 microns, for example.

Pores within the matrix may be fabricated in accordance with the principles described herein, for example, microelectromechanical (MEMS) technology, metal oxide semiconductor (CMOS) technology, micro-manufacturing processes for producing microsieves, laser technology, irradiation, molding, and micromachining, for example, or a combination thereof

The porous matrix is permanently attached to a holder which can be associated to the bottom of the liquid holder and to the top of the vacuum manifold where the porous matrix is positioned such that liquid can flow from liquid holder to vacuum manifold. In some examples, the porous matrix in the holder can be associated to a microfluidic surface, top or bottom cover surface. The holder may be constructed of any suitable material that is compatible with the material of the porous matrix. Examples of such materials include, by way of example and not limitation, any of the materials listed above for the porous matrix. The material for the housing and for the porous matrix may be the same or may be different. The holder may also be constructed of non-porous glass or plastic film.

Examples of plastic film materials include polystyrene, polyalkylene, polyolefins, epoxies, Teflon®, PET, chloro-fluoroethylenes, polyvinylidene fluoride, PE-TFE, PE-CTFE, liquid crystal polymers, Mylar®, polyester, polymethylpentene, polyphenylene sulfide, and PVC plastic films. The plastic film can be metallized such as with aluminum. The plastic films can have relative low moisture transmission rate, e.g. 0.001 mg per m²-day. The porous matrix may be permanently attached to a holder by adhesion using thermal bonding, mechanical fastening or through use of permanent adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein and lignin. The plastic film or the adhesive can be electrically conductive materials and the conductive material coatings or materials can be patterned across specific regions of the hold surface.

The porous matrix in the holder is generally part of a filtration module where the porous matrix is part of an assembly for convenient use during filtration. The holder does not contain pores and has a surface which facilitates contact with associated surfaces but is not permanently attached to these surfaces and can be removed. A top gasket maybe applied to the removable holder between the liquid holder. A bottom gasket maybe applied to the removable holder between the manifold for vacuum. A gasket is a flexible material that facilities complete contact upon compression. The holder maybe constructed of gasket material. Examples of gasket shapes include a flat, embossed, patterned, or molded sheets, rings, circles, ovals, with cut out areas to allow sample to flow from porous matrix to vacuum manifold. Examples of gasket materials include paper, rubber, silicone, metal, cork, felt, neoprene, nitrile rubber, fiberglass, polytetrafluoroethylene like PTFE or Teflon or a plastic polymer like polychlorotrifluoro-ethylene.

In some examples, vacuum is applied to the concentrated and treated sample on the porous matrix to facilitate passage of non-rare cells through the matrix. The level of vacuum applied is dependent on one or more of the nature and size of the different populations of biological particles, the nature of the porous matrix, and the size of the pores of the porous matrix, for example. In some examples, the level of vacuum applied is about 1 millibar to about 100 millibar, or about 1 millibar to about 80 millibar, or about 1 millibar to about 50 millibar, or about 1 millibar to about 40 millibar, or about 1 millibar to about 30 millibar, or about 1 millibar to about 25 millibar, or about 1 millibar to about 20 millibar, or about 1 millibar to about 15 millibar, or about 1 millibar to about 10 millibar, or about 5 millibar to about 80 millibar, or about 5 millibar to about 50 millibar, or about 5 millibar to about 30 millibar, or about 5 millibar to about 25 millibar, or about 5 millibar to about 20 millibar, or about 5 millibar to about 15 millibar, or about 5 millibar to about 10 millibar, for example. In some examples the vacuum is an oscillating vacuum, which means that the vacuum is applied intermittently at regular or irregular intervals, which may be, for example, about 1 second to about 600 seconds, or about 1 second to about 500 seconds, or about 1 second to about 250 seconds, or about 1 second to about 100 seconds, or about 1 second to about 50 seconds, or about 10 seconds to about 600 seconds, or about 10 seconds to about 500 seconds, or about 10 seconds to about 250 seconds, or about 10 seconds to about 100 seconds, or about 10 seconds to about 50 seconds, or about 100 seconds to about 600 seconds, or about 100 seconds to about 500 seconds, or about 100 seconds to about 250 seconds, for example. In this approach, vacuum is oscillated at about 0 millibar to about 10 millibar, or about 1 millibar to about 10 millibar, or about 1 millibar to about 7.5 millibar, or about 1 millibar to about 5.0 millibar, or about 1 millibar to about 2.5 millibar, for example, during some or all of the application of vacuum to the blood sample. Oscillating vacuum is achieved using an on-off switch, for example, and may be conducted automatically or manually.

Contact of the treated sample with the porous matrix is continued for a period of time sufficient to achieve retention of the rare cells or the particle-bound rare molecules on a surface of the porous matrix to obtain a surface of the porous matrix having different populations of rare cells or the particle-bound rare molecules as discussed above. The period of time is dependent on one or more of the nature and size of the different populations of rare cells or particle-bound rare molecules, the nature of the porous matrix, the size of the pores of the porous matrix, the level of vacuum applied to the sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

### Examples of Rare Molecules

The phrase “rare molecules” refers to a molecule that may be detected in a sample where the rare molecules is indicative of a particular population of molecules. The phrase “population of molecules” refers to a group of rare molecules that share common rare molecules that is specific for the group of rare molecules. The phrase “specific for” means that the common rare molecules distinguishes the group of rare molecules from other molecules.

The methods described herein involve trace analysis, i.e., minute amounts of material on the order of 1 to about 100,000 copies of rare cells or rare molecules. Since this process involves trace analysis at the detection limits of the nucleic acid analyzers, these minute amounts of material can only be detected when detection volumes are extremely low, for example, 10-15 liter, so that the concentrations are within the detection. Given associated errors is unlikely and that “all” of the rare molecules undergo amplification, i.e., converting the minute amounts of material to the order of about 10⁵ to about 10¹⁰ copies of every rare molecule. The phrase “substantially all” means that at least about 70 to about 99% measured by the reproducibility of the amount of a rare molecule produced.

The phrase “cell free rare molecules” refers to rare molecules that are not bound to a cell and/or that freely circulate in a sample. Such non-cellular rare molecules include biomolecules useful in medical diagnosis and treatments of diseases. Medical diagnosis of diseases include, but are not limited to, biomarkers for detection of cancer, cardiac damage, cardiovascular disease, neurological disease, hemostasis/hemastasis, fetal maternal assessment, fertility, bone status, hormone levels, vitamins, allergies, autoimmune diseases, hypertension, kidney disease, metabolic disease, diabetes, liver diseases, infectious diseases and other biomolecules useful in medical diagnosis of diseases, for example.

The following are non-limiting examples of samples that rare molecules that can be measured in. The sample to be analyzed is one that is suspected of containing rare molecules. The samples may be biological samples or non-biological samples. Biological samples may be from a plant, animal, protists or other living organism including Animalia, fungi, plantae, chromista, or protozoa or other eukaryote species or bacteria, archaea, or other prokaryote species. Non-biological samples include aqueous solutions, environmental, products, chemical reaction production, waste streams, foods, feed stocks, fertilizers, fuels, and the like. Biological samples include biological fluids such as whole blood, serum, plasma, sputum, lymphatic fluid, semen, vaginal mucus, feces, urine, spinal fluid, saliva, stool, cerebral spinal fluid, tears, mucus, or tissues for example. Biological tissue includes, by way of illustration, hair, skin, sections or excised tissues from organs or other body parts, for example. Rare molecules may be from tissues, for example, lung, bronchus, colon, rectum, extra cellular matrix, dermal, vascular, stem, lead, root, seed, flower, pancreas, prostate, breast, liver, bile duct, bladder, ovary, brain, central nervous system, kidney, pelvis, uterine corpus, oral cavity or pharynx or cancers. In many instances, the sample is aqueous such as a urine, whole blood, plasma or serum sample, in other instances the sample must be made into a solution or suspension for testing.

The sample can be one that contains cells such as, for example, non-rare cells and rare cells where rare molecules are detected from the rare cells. The rare molecules from cells may be from any organism, but are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesochymal cells; circulating epithelial cells; fetal cells; immune cells (B cells, T cells, macrophages, NK cells, monocytes); and stem cells; for example. In other examples of methods in accordance with the principles described herein, the sample to be tested is a blood sample from a organism such as, but not limited to, a plant or animal subject, for example. In some examples of methods in accordance with the principles described herein, the sample to be tested is a sample from a organism such as, but not limited to, a mammal subject, for example. Cells with rare molecules may be from a tissue of mammal, for example, lung, bronchus, colon, rectum, pancreas, prostate, breast, liver, bile duct, bladder, ovary, brain, central nervous system, kidney, pelvis, uterine corpus, oral cavity or pharynx or cancers.

Rare molecule fragments can be used to measure peptidases of interest including those in the MEROPS is an on-line database for peptidases (also known as proteases) and total ˜902212 different sequences of aspartic, cysteine, glutamic, metallo, asparagine, serine, threonine and general peptidases catalytics types which are further categorized and include those listed for the following pathways: 2-Oxocarboxylic acid metabolism, ABC transporters, African trypano-somiasis, Alanine, aspartate and glutamate metabolism, Allograft rejection, Alzheimer's disease, Amino sugar and nucleotide sugar metabolism, Amoebiasis, AMPK signaling pathway, Amyotrophic lateral sclerosis (ALS), Antigen processing and presentation, Apoptosis, Arachidonic acid metabolism, Arginine and proline metabolism, Arrhythmogenic right ventricular cardiomyopathy (ARVC), Asthma, Autoimmune thyroid disease, B cell receptor signaling pathway, Bacterial secretion system, Basal transcription factors, beta-Alanine metabolism, Bile secretion, Biosynthesis of amino acids, Biosynthesis of secondary metabolites, Biosynthesis of unsaturated fatty acids, Biotin metabolism, Bisphenol degradation, Bladder cancer, cAMP signaling pathway, Carbon metabolism, Cardiac muscle contraction, Cell adhesion molecules (CAMs), Cell cycle, Cell cycle—yeast, Chagas disease (American trypano-somiasis), Chemical carcinogenesis, Cholinergic synapse, Colorectal cancer, Complement and coagulation cascades, Cyanoamino acid metabolism, Cysteine and methionine metabolism, Cytokine-cytokine receptor interaction, Cytosolic DNA-sensing pathway, Degradation of aromatic compounds, Dilated cardiomyopathy, Dioxin degradation, DNA replication, Dorso-ventral axis formation, Drug metabolism—other enzymes, Endocrine and other factor-regulated calcium reabsorption, Endocytosis, Epithelial cell signaling in Helicobacter pylori infection, Epstein-Barr virus infection, Estrogen signaling pathway, Fanconi anemia pathway, Fatty acid elongation, Focal adhesion, Folate biosynthesis, FoxO signaling pathway, Glutathione metabolism, Glycerolipid metabolism, Glycerophospholipid metabolism, Glycosylphosphatidyl-inositol(GPI)-anchor biosynthesis, Glyoxylate and dicarboxylate metabolism, GnRH signaling pathway, Graft-versus-host disease, Hedgehog signaling pathway, Hematopoietic cell lineage, Hepatitis B, Herpes simplex infection, HIF-1 signaling pathway, Hippo signaling pathway, Histidine metabolism, Homologous recombination, HTLV-I infection, Huntington's disease, Hypertrophic cardiomyopathy (HCM), Influenza A, Insulin signaling pathway, Legionellosis, Leishmaniasis, Leukocyte transendothelial migration, Lysine biosynthesis, Lysosome, Malaria, MAPK signaling pathway, Meiosis—yeast, Melanoma, Metabolic pathways, Metabolism of xenobiotics by cytochrome P450, Microbial metabolism in diverse environments, MicroRNAs in cancer, Mineral absorption, Mismatch repair, Natural killer cell mediated cytotoxicity, Neuroactive ligand-receptor interaction, NF-kappa B signaling pathway, Nitrogen metabolism, NOD-like receptor signaling pathway, Non-alcoholic fatty liver disease (NAFLD), Notch signaling pathway, Olfactory transduction, Oocyte meiosis, Osteoclast differentiation, Other glycan degradation, Ovarian steroidogenesis, Oxidative phosphorylation, p53 signaling pathway, Pancreatic secretion, Pantothenate and CoA biosynthesis, Parkinson's disease, Pathways in cancer, Penicillin and cephalosporin biosynthesis, Peptidoglycan biosynthesis, Peroxisome, Pertussis, Phagosome, Phenylalanine metabolism, Phenylalanine, tyrosine and tryptophan biosynthesis, Phenylpropanoid biosynthesis, PI3K-Akt signaling pathway, Plant-pathogen interaction, Platelet activation, PPAR signaling pathway, Prion diseases, Proteasome, Protein digestion and absorption, Protein export, Protein processing in endoplasmic reticulum, Proteoglycans in cancer, Purine metabolism, Pyrimidine metabolism, Pyruvate metabolism, Rap1 signaling pathway, Ras signaling pathway, Regulation of actin cytoskeleton, Regulation of autophagy, Renal cell carcinoma, Renin-angiotensin system, Retrograde endocannabinoid signaling, Rheumatoid arthritis, RIG-I-like receptor signalling pathway, RNA degradation, RNA transport, Salivary secretion, Salmonella infection, Serotonergic synapse, Small cell lung cancer, Spliceosome, Staphylococcus aureus infection, Systemic lupus erythematosus, T cell receptor signaling pathway, Taurine and hypotaurine metabolism, Terpenoid backbone biosynthesis, TGF-beta signaling pathway, TNF signaling pathway, Toll-like receptor signaling pathway, Toxoplasmosis, Transcriptional misregulation in cancer, Tryptophan metabolism, Tuberculosis, Two-component system, Type I diabetes mellitus, Ubiquinone and other terpenoid-quinone bio-synthesis, Ubiquitin mediated proteolysis, Vancomycin resistance, Viral carcinogenesis, Viral myocarditis, Vitamin digestion and absorption Wnt signaling pathway.

Rare molecule fragments can be used to measure peptidase inhibitor of interest included those in the MEROPS on-line database for peptidase inhibitors and include and total ˜133535 different sequences of where a family is a set of homologous peptidase inhibitors with a homology. The homology is shown by a significant similarity in amino acid sequence either to the type inhibitor of the family, or to another protein that has already been shown to be homologous to the type inhibitor, and thus a member of. The reference organism for the family includes ovomucoid inhibitor unit 3 (Meleagris gallopavo)aprotinin (Bos taurus), soybean Kunitz trypsin inhibitor (Glycine max), proteinase inhibitor B (Sagittaria sagittifolia), alpha-1-peptidase inhibitor (Homo sapiens), ascidian trypsin inhibitor (Halocynthia roretzi), ragi seed trypsin/alpha-amylase inhibitor (Eleusine coracana), trypsin inhibitor MCTI-1 (Momordica charantia), Bombyx subtilisin inhibitor (Bombyx mori), peptidase B inhibitor (Saccharomyces cerevisiae), marinostatin (Alteromonas sp.), ecotin (Escherichia coli), Bowman-Birk inhibitor unit 1 (Glycine max), eglin c (Hirudo medicinalis), hirudin (Hirudo medicinalis), antistasin inhibitor unit 1 (Haementeria officinalis), streptomyces subtilisin inhibitor (Streptomyces albogriseolus), secretory leukocyte peptidase inhibitor domain 2 (Homo sapiens), mustard trypsin inhibitor-2 (Sinapis alba), peptidase inhibitor LMPI inhibitor unit 1 (Locusta migratoria), potato peptidase inhibitor II inhibitor unit 1 (Solanum tuberosum), secretogranin V (Homo sapiens), BsuPI peptidase inhibitor (Bacillus subtilis), pinA Lon peptidase inhibitor (Enterobacteria phage T4), cystatin A (Homo sapiens), ovocystatin (Gallus gallus), metallo-peptidase inhibitor (Bothrops jararaca), calpastatin inhibitor unit 1 (Homo sapiens), cytotoxic T-lymphocyte antigen-2 alpha (Mus musculus), equistatin inhibitor unit 1 (Actinia equina), survivin (Homo sapiens), aspin (Ascaris suum), saccharopepsin inhibitor (Saccharomyces cerevisiae), timp-1 (Homo sapiens), Streptomyces metallopeptidase inhibitor (Streptomyces nigrescens), potato metallocarboxypeptidase inhibitor (Solanum tuberosum), metallopeptidase inhibitor (Dickeya chrysanthemi), alpha-2-macroglobulin (Homo sapiens), chagasin (Leishmania major), oprin (Didelphis marsupialis), metallocarboxypeptidase A inhibitor (Ascaris suum), leech metallocarboxypeptidase inhibitor (Hirudo medicinalis), latexin (Homo sapiens), clitocypin (Lepista nebularis), proSAAS (Homo sapiens), baculovirus P35 caspase inhibitor (Spodoptera litura nucleopolyhedrovirus), p35 homologue (Amsacta moorei entomopoxvirus), serine carboxypeptidase Y inhibitor (Saccharomyces cerevisiae), tick anticoagulant peptide (Ornithodoros moubata), madanin 1 (Haemaphysalis longicornis), squash aspartic peptidase inhibitor (Cucumis sativus), staphostatin B (Staphylococcus aureus), staphostatin A (Staphylococcus aureus), triabin (Triatoma pallidipennis), pro-eosinophil major basic protein (Homo sapiens), thrombostasin (Haematobia irritans), Lentinus peptidase inhibitor (Lentinula edodes), bromein (Ananas comosus), tick carboxypeptidase inhibitor (Rhipicephalus bursa), streptopain inhibitor (Streptococcus pyogenes), falstatin (Plasmodium falciparum), chimadanin (Haemaphysalis longicornis), {Veronica} trypsin inhibitor (Veronica hederifolia), variegin (Amblyomma variegatum), bacteriophage lambda CIII protein (bacteriophage lambda), thrombin inhibitor (Glossina morsitans), anophelin (Anopheles albimanus), Aspergillus elastase inhibitor (Aspergillus fumigatus), AVR2 protein (Passalora fulva), IseA protein (Bacillus subtilis), toxostatin-1 (Toxoplasma gondii), AmFPI-1 (Antheraea mylitta), cvSl-2 (Crassostrea virginica), macrocypin 1 (Macrolepiota procera), HflC (Escherichia coli), oryctin (Oryctes rhinoceros), trypsin inhibitor (Mirabilis jalapa), F1L protein (Vaccinia virus), NvCI carboxypeptidase inhibitor (Nerita versicolor), Sizzled protein (Xenopus laevis), EAPH2 protein (Staphylococcus aureus), and Bowman-Birk-like trypsin inhibitor (Odorrana versabilis). Rare molecule fragments can be used to measure synthetic inhibition of peptidase inhibitor. The aforementioned data base also includes examples of thousands of different small molecule inhibitors that can mimic the inhibitory properties for any member or the above listed family.

Rare molecules of metabolic interest include but are not limited to those that impact the concentration of ACC Acetyl Coenzyme A Carboxylase, Adpn Adiponectin, AdipoR Adiponectin Receptor, AG Anhydroglucitol, AGE Advance glycation end products, Akt Protein kinase B, AMBK pre-alpha-l-microglobulin/bikunin, AMPK 5′-AMP activated protein kinase, ASP Acylation stimulating protein, Bik Bikunin, BNP B-type natriuretic peptide, CCL Chemokine (C-C motif) ligand, CINC Cytokine-induced neutrophil chemoattractant, CTF C-Terminal Fragment of Adiponectin Receptor, CRP C-reactive protein, DGAT Acyl CoA diacyl-glycerol transferase, DPP-IV Dipeptidyl peptidase-IV, EGF Epidermal growth factor, eNOS Endothelial NOS, EPO Erythropoietin, ET Endothelin, Erk Extracellular signal-regulated kinase, FABP Fatty acid-binding protein, FGF Fibroblast growth factor, FFA Free fatty acids, FXR Farnesoid X receptor a, GDF Growth differentiation factor, GH Growth hormone, GIP Glucose-dependent insulinotropic polypeptide, GLP Glucagon-like peptide-1, GSH Glutathione, GHSR Growth hormone secretagogue receptor, GULT Glucose transporters, GCD59 glycated CD59 (aka glyCD59), HbA1c Hemogloblin A1c, HDL High-density lipoprotein, HGF Hepatocyte growth factor, HIF Hypoxia-inducible factor, HMG 3-Hydroxy-3-methylglutaryl CoA reductase, I-α-I Inter-α-inhibitor, Ig-CTF Immunoglobulin attached C-Terminal Fragment of AdipoR, insulin, IDE Insulin-degrading enzyme, IGF Insulin-like growth factor, IGFBP IGF binding proteins, IL Interleukin cytokines, ICAM Intercellular adhesion molecule, JAK STAT Janus kinase/signal transducer and activator of transcription, JNK c-Jun N-terminal kinases, KIM Kidney injury molecule, LCN-2 Lipocalin,LDL Low-density lipoprotein, L-FABP Liver type fatty acid binding protein, LPS Lipopolysaccharide, Lp-PLA2 Lipoprotein-associated phospho-lipase A2, LXR Liver X receptors, LYVE Endothelial hyaluronan receptor, MAPK Mitogen-activated protein kinase, MCP Monocyte chemotactic protein, MDA Malondialdehyde, MIC Macrophage inhibitory cytokine, MIP Macrophage infammatory protein, MMP Matrix metallo-proteinase, MPO Myeloperoxidase, mTOR Mammalian of rapamycin, NADH Nicotinamide adenine dinucleotide, NGF Nerve growth factor, NFKB Nuclear factor kappa-light-chain-enhancer of activated B cells, NGAL Neutrophil gelatinase lipocalin, NOS Nitric oxide synthase NOX NADPH oxidase NPY Neuropeptide Yglucose, insulin, proinsulin, c peptide OHdG Hydroxydeoxyguanosine, oxLDL Oxidized low density lipoprotein, P-α-I pre-interleukin-α-inhibitor, PAI-1 Plasminogen activator inhibitor, PAR Protease-activated receptors, PDF Placental growth factor, PDGF Platelet-derived growth factor, PKA Protein kinase A, PKC Protein kinase C, PI3K Phosphatidylinositol 3-kinase, PLA2 Phosphatidylinositol 3-kinase, PLC Phospholipase C, PPAR Peroxisome proliferator-activated receptor, PPG Postprandial glucose, PS Phosphatidylserine, PR Protienase, PYY Neuropeptide like peptide Y, RAGE Receptors for AGE, ROS Reactive oxygen species, 5100 Calgranulin, sCr Serum creatinine, SGLT2 Sodium-glucose transporter 2, SFRP4 secreted frizzled-related protein 4 precursor, SREBP Sterol regulatory element binding proteins, SMAD Sterile alpha motif domain-containing protein, SOD Superoxide dismutase, sTNFR Soluble TNF α receptor, TACE TNFα alpha cleavage protease, TFPI Tissue factor pathway inhibitor, TG Triglycerides, TGF β Transforming growth factorβ, TIMP Tissue inhibitor of metalloproteinases, TNF α Tumor necrosis factorsα, TNFR TNF α receptor, THP Tamm-Horsfall protein, TLR Toll-like receptors, TnI Troponin I, tPA Tissue plasminogen activator, TSP Thrombospondin, Uri Uristatin, uTi Urinary trypsin inhibitor, uPA Urokinase-type plasminogen activator, uPAR uPA receptor, VCAM Vascular cell adhesion molecule, VEGF Vascular endothelial growth factor, and YKL-40 Chitinase-3-like protein.

Rare molecules of interest that are highly expressed by pancreas include but are not limited to INS insulin, GLU gluogen, NKX6-1 transcription factor, PNLIPRP1 pancreatic lipase-related protein 1, SYCN syncollin, PRSS1 protease, serine, 1 (trypsin 1) Intracellular, CTRB2 chymotrypsinogen B2 Intracellular, CELA2A chymotrypsin-like elastase family, member 2A, CTRB1 chymotrypsinogen B1 Intracellular,CELA3A chymotrypsin-like elastase family, member 3A Intracellular, CELA3B chymotrypsin-like elastase family, member 3B Intracellular, CTRC chymotrypsin C (caldecrin), CPA1 carboxypeptidase A1 (pancreatic) Intracellular, PNLIP pancreatic lipase, and CPB1 carboxypeptidase B1 (tissue), AMY2A amylase, alpha 2A (pancreatic), and CTFR cystic fibrosis transmembrane conductance regulator. Rare molecule fragments include those of insulin generated by the following peptidases known to naturally act on insulin; archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, matrix metallopeptidase-9 and others. These fragments include but are not limited to the following sequences: SEQ ID NO: 1 MALWMRLLPLLALLALWGP, SEQ ID NO: 2 MALWMRLL-PL, SEQ ID NO: 3 ALLALWGPD, SEQ ID NO: 4 AAAFVNQHLCGSHLVEALYLVCGERGF-FYTPKTR, SEQ ID NO: 5 PAAAFVNQHLCGSHLVEALYLVC, SEQ ID NO: 6 PAAAF-VNQHLCGS, SEQ ID NO: 7 CGSHLVEALYLV, SEQ ID NO: 8 VEALYLVC, SEQ ID NO: 9 LVCGERGF, SEQ ID NO: 10 FFYTPK, SEQ ID NO: 11 REAEDLQVGQVELGGGPGA-GSLQPLALEGSL, SEQ ID NO: 12 REAEDLQVGQVE, SEQ ID NO: 13 LGGGPGAG, SEQ ID NO: 14 SLQPLALEGSL, SEQ ID NO: 15 GIVEQCCTSICSLYQLENYCN, SEQ ID NO: 16 GIVEQCCTSICSLY, SEQ ID NO: 17 QLENYCN, AND SEQ ID NO: 18 CSLYQLE variation within 75% exact homology. Variations include natural and modified aminoacids.

The rare molecule fragments of insulin can be used to measure the peptidases acting on insulin based on formation of fragments. This includes the list of natural known peptidase and others added to the biological system. Additional rare molecule fragments of insulin of can be used to measure inhibitor for peptidases acting on insulin peptidases based on the lack formation of fragments. These inhibitor include the c-Terminal fragment of the Adiponectin Receptor, Bikunin, Uristatin and other known natural and synthetic inhibitors of archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, and matrix metallopeptidase-9 listed in the inhibitor databases.

Rare molecule fragments examples of bioactive proteins and peptides which can be used to measure presents or absence thereof as an indication of therapeutic effectiveness, stability, usage, metabolism, action on biological pathways (such as actions with proteases, peptidase, enzymes, receptors or other biomolecules), action of inhibition of pathways and other interactions with biological systems. Examples include but are not limited to those list in databases of approved therapeutic peptides and proteins, such as http://crdd.osdd.net/ as well as other databases of peptides and proteins for dietary supplements, probiotics, food safety, veterinary products, and cosmetics usage. The list of the 467 approved peptide and protein therapies include examples of bioactive proteins and peptides for use in cancer, metabolic disorders, hematological disorders, immunological disorders, genetic disorders, hormonal disorders, bone disorders, cardiac disorders, infectious disease, respiratory disorders, neurological disorders, adjunct therapy, eye disorders, and malabsorption disorder. Bioactive proteins and peptides include those used as anti-thrombins, fibrinolytic, enzymes, antineoplastic agents, hormones, fertility agents, immunosupressive agents, bone related agents, antidiabetic agents, and antibodies

Some specific examples of therapeutic proteins and peptides include glucagon, ghrelin, leptin, growth hormone, prolactin, human placental, lactogen, luteinizing hormone, follicle stimulating hormone, chorionic gonadotropin, thyroid stimulating hormone, adrenocorticotropic hormone, vasopressin, oxytocin, angiotensin, parathyroid hormone, gastrin, buserelin, antihemophilic factor, pancrelipase, insulin, insulin aspart, porcine insulin, insulin lispro, insulin isophane, insulin glulisine, insulin detemir, insulin glargine, immunglobulins, interferon, leuprolide, denileukin, asparaginase, thyrotropin, alpha-1-proteinase inhibitor, exenatide, albumin, coagulation factors, alglucosidase alfa, salmon calcitonin, vasopressin, epidermal growth factor (EGF), cholecystokinin (CCK-8), vacines, human growth hormone and others.

Some new examples of therapeutic proteins and peptides include GLP-1-GCG, GLP-1-GIP, GLP-1, GLP-1- GLP-2, and GLP-1-CCKB

Rare molecules of interest that are highly expressed by adipose tissue include but are not limited to ADIPOQ Adiponectin, C1Q and collagen domain containing, TUSC5 Tumor suppressor candidate 5, LEP Leptin, CIDEA Cell death-inducing DFFA-like effector a, CIDEC Cell death-inducing DFFA-like effector C, FABP4 Fatty acid binding protein 4, adipocyte, LIPE, GYG2, PLIN1 Perilipin 1, PLIN4 Perilipin 4, CSN1S1, PNPLA2, RP11-407P15.2 Protein LOC100509620, L GALS12 Lectin, galactoside-binding, soluble 12, GPAM Glycerol-3-phosphate acyltransferase, mitochondrial, PR325317.1 predicted protein, ACACB Acetyl-CoA carboxylase beta, ACVR1C Activin A receptor, type IC, AQP7 Aquaporin 7, CFD Complement factor D (adipsin)m CSN1S1Casein alpha s1, FASN Fatty acid synthase GYG2 Glycogenin 2 KIF25Kinesin family member 25 LIPELipase, hormone-sensitive PNPLA2, Patatin-like phospholipase domain containing 2 SLC29A4, Solute label family 29 (equilibrative nucleoside transporter), member 4 SLC7A10 Solute label family 7 (neutral amino acid transporter light chain, asc system), member 10, SPX Spexin hormone and TIMP4 TIMP metallopeptidase inhibitor 4.

Rare molecules of interest that are highly expressed by adrenal gland and thyroid include but are not limited to CYP11B2 Cytochrome P450, family 11, subfamily B, polypeptide 2, CYP11B1 Cytochrome P450, family 11, subfamily B, polypeptide 1, CYP17A1 Cytochrome P450, family 17, subfamily A, polypeptide 1, MC2R Melanocortin 2 receptor (adrenocorticotropic hormone), CYP21A2 Cytochrome P450, family 21, subfamily A, poly-peptide 2, HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-mono-oxygenase), HSD3B2 Hydroxy-delta-5-steroid dehydrogenase, 3 beta- and steroid delta-iso-merase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), AKR1B1 Aldo-keto reductase family 1, member B1 (aldose reductase), NOV Nephroblastoma overexpressed, FDX1 Ferredoxin 1, DGKK Diacylglycerol kinase, kappa, MGARP Mitochondria-localized glutamic acid-rich protein, VWA5B2 Von Willebrand factor A domain containing 5B2, C18orf42 Chromosome 18 open reading frame 42, KIAA1024, MAP3K15 Mitogen-activated protein kinase kinase kinase 15, STAR Steroidogenic acute regulatory protein Potassium channel, subfamily K, member 2, NOV nephroblastoma overexpressed, PNMT phenylethanolamine N-methyltransferase, CHGB chromogranin B (secretogranin 1), and PHOX2A paired-like homeobox 2a.

Rare molecules of interest that are highly expressed by bone marrow include but are not limited to DEFA4 defensin alpha 4 corticostatin, PRTN3 proteinase 3, AZU1 azurocidin 1, DEFA1 defensin alpha 1, ELANE elastase, neutrophil expressed, DEFA1B defensin alpha 1B, DEFA3 defensin alpha 3 neutrophil-specific, MS4A3 membrane-spanning 4-domains, subfamily A, member 3 (hematopoietic cell-specific), RNASE3 ribonuclease RNase A family 3, MPO myeloperoxidase, HBD hemoglobin, delta, and PRSS57 protease, serine 57.

Rare molecules of interest that are highly expressed by the brain include but are not limited to GFAP glial fibrillary acidic protein, OPALIN oligodendrocytic myelin paranodal and inner loop protein, OLIG2 oligodendrocyte lineage transcription factor 2, GRIN1glutamate receptor ionotropic, N-methyl D-aspartate 1, OMG oligodendrocyte myelin glycoprotein, SLC17A7 solute label family 17 (vesicular glutamate transporter), member 7, C1 orf61 chromosome 1 open reading frame 61, CREG2 cellular repressor of E1A-stimulated genes 2, NEUROD6 neuronal differentiation 6, ZDHHC22 zinc finger DHHC-type containing 22, VSTM2B V-set and transmembrane domain containing 2B, and PMP2 peripheral myelin protein 2.

Rare molecules of interest that are highly expressed by the endometrium, ovary, or placenta include but are not limited to MMP26 matrix metallopeptidase 26, MMP10 matrix metallopeptidase 10 (stromelysin 2), RP4-559A3.7 uncharacterized protein and TRH thyro-tropin-releasing hormone.

Rare molecules of interest that are highly expressed by gastrointestinal tract, salivary gland, esophagus, stomach, duodenum, small intestine, or colon include but are not limited to GKN1 Gastrokine 1, GIF Gastric intrinsic factor (vitamin B synthesis) , PGAS Pepsinogen 5 group I (pepsinogen A), PGA3 Pepsinogen 3, group I (pepsinogen A, PGA4 Pepsinogen 4 group I (pepsinogen A), LCT Lactase, DEFAS Defensin, alpha 5 Paneth cell-specific, CCL25 Chemokine (C-C motif) ligand 25, DEFA6 Defensin alpha 6 Paneth cell-specific, GAST Gastrin, MS4A10 Membrane-spanning 4-domains subfamily A member 10, ATP4A and ATPase, H+/K+exchanging alpha polypeptide

Rare molecules of interest that are highly expressed by heart or skeletal muscle include but are not limited to NPPB natriuretic peptide B, TNNI3 troponin I type 3 (cardiac), NPPA natriuretic peptide A, MYL7 myosin light chain 7 regulatory, MYBPC3 myosin binding protein C (cardiac), TNNT2 troponin T type 2 (cardiac) LRRC10 leucine rich repeat containing 10, ANKRD1 ankyrin repeat domain 1 (cardiac muscle), RD3L retinal degeneration 3-like, BMP10 bone morphogenetic protein 10 , CHRNE cholinergic receptor nicotinic epsilon (muscle), and SBK2 SH3 domain binding kinase family member 2.

Rare molecules of interest that are highly expressed by kidney include but are not limited to UMOD uromodulin, TMEM174 transmembrane protein 174, SLC22A8 solute label family 22 (organic anion transporter) member 8, SLC12A1 solute label family 12 (sodium/potassium/chloride transporter) member 1, SLC34A1 solute label family 34 (type II sodium/phosphate transporter) member 1, SLC22Al2 solute label family 22 (organic anion/urate transporter) member 12, SLC22A2 solute label family 22 (organic cation transporter) member 2, MCCD1 mitochondrial coiled-coil domain 1, AQP2 aquaporin 2 (collecting duct), SLC7A13 solute label family 7 (anionic amino acid transporter) member 13, KCNJ1 potassium inwardly-rectifying channel, subfamily J member 1 and SLC22A6 solute label family 22 (organic anion transporter) member 6.

Rare molecules of interest that are highly expressed by lung include but are not limited to SFTPC surfactant protein C, SFTPA1 surfactant protein A1, SFTPB surfactant protein B, SFTPA2 surfactant protein A2, AGER advanced glycosylation end product-specific receptor, SCGB3A2 secretoglobin family 3A member 2, SFTPD surfactant protein D, ROS1 proto-oncogene 1 receptor tyrosine kinase, MS4A15 membrane-spanning 4-domains subfamily A member 15, RTKN2 rhotekin 2, NAPSA napsin A aspartic peptidase, and LRRN4 leucine rich repeat neuronal 4.

Rare molecules of interest that are highly expressed by liver or gallbladder include but are not limited to APOA2 apolipoprotein A-II, A1BG alpha-1-B glycoprotein, AHSG alpha-2-HS-glycoprotein, F2coagulation factor II (thrombin), CFHR2 complement factor H-related 2, HPX hemopexin, F9 coagulation factor IX, CFHR2 complement factor H-related 2, SPP2 secreted phosphoprotein 2 (24kDa), C9 complement component 9, MBL2 mannose-binding lectin (protein C) 2 soluble and CYP2A6 cytochrome P450 family 2 subfamily A polypeptide 6.

Rare molecules of interest that are highly expressed by testis or prostate include but are not limited to PRM2 protamine 2, PRM1 protamine 1, TNP1 transition protein 1 (during histone to protamine replacement) TUBA3C tubulin, alpha 3c LELP1 late cornified envelope-like proline-rich 1, BOD1L2 biorientation of chromosomes in cell division 1-like 2 ANKRD7 ankyrin repeat domain 7, PGK2 phosphoglycerate kinase 2, AKAP4 A kinase (PRKA) anchor protein 4, TPD52L3 tumor protein D52-like 3, UBQLN3 ubiquilin 3, and ACTL7A actin-like 7A.

### Examples of Rare Cells and Rare Cell Markers

Rare cells are those cells that are present in a sample in relatively small quantities when compared to the amount of non-rare cells in a sample. In some examples, the rare cells are present in an amount of about 10⁻⁸% to about 10⁻²% by weight of a total cell population in a sample suspected of containing the rare cells. The phrase “cell rare molecules” refers to rare molecules that are bound in a cell and may or may not freely circulate in a sample. Such cellular rare molecule include biomolecules useful in medical diagnosis of diseases as above and also include all rare molecules and uses previously described in for cell free rare molecules and those for biomolecules used for measurement of rare cells. The rare cells (cell markers) may be, but are not limited to, malignant cells such as malignant neoplasms or cancer cells; circulating cells, endothelial cells (CD146); epithelial cells (CD326/EpCAM); mesenchymal cells (VIM), bacterial cells, virus, skin cells, sex cells, fetal cells; immune cells (leukocytes such as basophil, granulocytes (CD66b) and eosinophil, lymphocytes such as B cells (CD19,CD20), T cells (CD3, CD4, CD8), plasma cells, and NK cells (CD56), macrophages/monocytes (CD14, CD33), dendritic cells (CD11c, CD123), Treg cells and others), stem cells/precursor (CD34), other blood cells such as progenitor, blast, erythrocytes, thrombocytes, platelets (CD41, CD61, CD62) and immature cells; other cells from tissues such as liver, brain, pancreas, muscle, fat, lung, prostate, kidney, urinary tract, adipose, bone marrow, endometrium, gastrointestinal tract, heart, testis or other for example.

The phrase “population of cells” refers to a group of cells having an antigen or nucleic acid on their surface or inside the cell where the antigen is common to all of the cells of the group and where the antigen is specific for the group of cells. Non-rare cells are those cells that are present in relatively large amounts when compared to the amount of rare cells in a sample. In some examples, the non-rare cells are at least about 10 times, or at least about 10² times, or at least about 10³ times, or at least about 10⁴ times, or at least about 10⁵ times, or at least about 10⁶ times, or at least about 10⁷ times, or at least about 10⁸ times greater than the amount of the rare cells in the total cell population in a sample suspected of containing non-rare cells and rare cells. The non-rare cells may be, but are not limited to, white blood cells, platelets, and red blood cells, for example.

The term “Rare cells markers” include, but are not limited to, cancer cell type biomarkers, cancer bio markers , chemo resistance biomarkers, metastatic potential biomarkers, and cell typing markers, cluster of differentiation (cluster of designation or classification determinant) (often abbreviated as CD) is a protocol used for the identification and investigation of cell surface molecules providing targets for immunophenotyping of cells, for example. Cancer cell type biomarkers include, by way of illustration and not limitation, cytokeratins (CK) (CK1, CK2, CK3, CK4, CKS, CK6, CK7, CK8 and CK9, CK10, CK12, CK 13, CK14, CK16, CK17, CK18, CK19 and CK2), epithelial cell adhesion molecule (EpCAM), N-cadherin, E-cadherin and vimentin, for example. Oncoproteins and oncogenes with likely therapeutic relevance due to mutations include, but are not limited to, WAF, BAX-1, PDGF, JAGGED 1, NOTCH, VEGF, VEGHR, CA1X, MIB1, MDM, PR, ER, SELS, SEMI, PI3K, AKT2, TWIST1, EML-4, DRAFF, to C-MET, ABL1, EGFR, GNAS, MLH1, RET, MEK1, AKT1, ERBB2, HER2, HNF1A, MPL, SMAD4, ALK, ERBB4, HRAS, NOTCH1, SMARCB1, APC, FBXW7, IDH1, NPM1, SMO, ATM, FGFR1, JAK2, NRAS, SRC, BRAF, FGFR2, JAK3, RA, STK11, CDH1, FGFR3, KDR, PIK3CA, TP53, CDKN2A, FLT3, KIT, PTEN, VHL, CSF1R, GNA11, KRAS, PTPN11, DDR2, CTNNB1, GNAQ, MET, RB1, AKT1, BRAF, DDR2, MEK1, NRAS, FGFR1, and ROS1, for example.

In certain embodiments, the rare cells may be endothelial cells which are detected using markers, by way of illustration and not limitation, CD136, CD105/Endoglin, CD144/VE-cadherin, CD145, CD34, Cd41 CD136, CD34, CD90, CD31/PECAM-1, ESAM,VEGFR2/Fik-1, Tie-2, CD202b/TEK, CD56/NCAM, CD73/VAP-2, claudin 5, Z0-1, and vimentin. Metastatic potential biomarkers include, but are limited to, urokinase plasminogen activator (uPA), tissue plasminogen activator (tPA), C terminal fragment of adiponectin receptor (Adiponectin Receptor C Terminal Fragment or Adiponectin CTF), kinases (AKT-PIK3, MAPK), vascular adhesion molecules (e.g., ICAM, VCAM, E-selectin), cytokine signaling (TNF-α, IL-1, IL-6), reactive oxidative species (ROS), protease-activated receptors (PARs), metalloproteinases (TIMP), transforming growth factor (TGF), vascular endothelial growth factor (VEGF), endothelial hyaluronan receptor 1 (LYVE-1), hypoxia-inducible factor (HIF), growth hormone (GH), insulin-like growth factors (IGF), epidermal growth factor (EGF), placental growth factor (PDF), hepatocyte growth factor (HGF), nerve growth factor (NGF), platelet-derived growth factor (PDGF), growth differentiation factors (GDF), VEGF receptor (soluble Flt-1), microRNA (MiR-141), Cadherins (VE, N, E), S100 Ig-CTF nuclear receptors (e.g., PPARa), plasminogen activator inhibitor (PAI-1), CD95, serine proteases (e.g., plasmin and ADAM, for example); serine protease inhibitors (e.g., Bikunin); matrix metalloproteinases (e.g., MMP9); matrix metalloproteinase inhibitors (e.g., TIMP-1); and oxidative damage of DNA.

Chemoresistance biomarkers include, by way of illustration and not limitation, PL2L piwi like, 5T4, ADLH, β-integrin, α-6-integrin, c-kit, c-met, LIF-R, chemokines (e.g., CXCR7,CCR7, CXCR4), ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24.

The rare molecules from cells may be from any organism, but are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesochymal cells; circulating epithelial cells; fetal cells; immune cells (B cells, T cells, macrophages, NK cells, monocytes); and stem cells; for example. In some examples of methods in accordance with the principles described herein, the sample to be tested is a blood sample from a mammal such as, but not limited to, a human subject, for example.

Rare cells of interest may be immune cells and include but are not limited to markers for white blood cells (WBC), Tregs (regulatory T cells), B cell, T cells, macrophages, monocytes, antigen presenting cells (APC), dendritic cells, eosinophils, and granulocytes. For example, markers such as, but not limited to, CD3, CD4, CD8, CD11 c, CD14, CD15, CD16, CD19, CD20, CD31, CD33, CD45, CD52, CD56, CD 61, CD66b, CD123, CTLA-4, immunoglobulin, protein receptors and cytokine receptors and other CD marker that are present on white blood cells can be used to indicate that a cell is not a rare cell of interest.

In a particular non-limiting examples white blood cell markers include CD45 antigen (also known as protein tyrosine phosphatase receptor type C or PTPRC) and originally called leukocyte common antigen is useful in detecting all white blood cells. Additionally, CD45 can be used to differentiate different types of white blood cells that might be considered rare cells. For example, granulocytes are indicated by CD45+, CD15+, or CD16+, or CD66b+; monocytes are indicated by CD45+, CD14+; T lymphocytes are indicated by CD45+, CD3+; T helper cells are indicated by CD45+,CD3+, CD4+; cytotoxic T cells are indicated by CD45+,CD3+, CDS+; B-lymphocytes are indicated by CD45+, CD19+or CD45+, CD20+; thrombocytes are indicated by CD45+, CD61+; and natural killer cells are indicated by CD16+, CD56+, and CD3-.

Furthermore, two commonly used CD molecules, namely, CD4 and CD8, are, in general, used as markers for helper and cytotoxic T cells, respectively. These molecules are defined in combination with CD3+, as some other leukocytes also express these CD molecules (some macrophages express low levels of CD4; dendritic cells express high levels of CD11c, and CD123. These examples are not inclusive of all marker and are for example only.

In some cases, the rare molecule fragment of lymphocytes include proteins and peptides produced as part of lymphocytes such as immunoglobulin chains, major histocompatibility complex (MHC) molecules, T cell receptors, antigenic peptides, cytokines, chemokines and their receptors (e.g, Interleukins, C-X-C chemokine receptors, etc), programmed death-ligand and receptors (Fas, PDL1, and others) and other proteins and peptides that are either parts of the lymphocytes or bind to the lymphocytes.

In other cases the rare cell maybe a stem cell and include but are not limited to the rare molecule fragment of stem markers cells including, PL2L piwi like, 5T4, ADLH, β-integrin, a6 integrin, c-kit, c-met, LIF-R, CXCR4, ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24. Stem cell markers include common pluripotency markers like FoxD3, E-Ras, Sa114, Stat3, SUZ12, TCF3, TRA-1-60, CDX2, DDX4, Miwi, Mill GCNF, Oct4, Klf4, Sox2,c-Myc, TIF 1 Piwil, nestin, integrin, notch, AML, GATA, Esrrb, Nr5a2, C/EBPα, Lin28, Nanog, insulin, neuroD, adiponectin, apdiponectin receptor, FABP4, PPAR, and KLF4 and the like.

In other cases the rare cell maybe a pathogen, bacteria, or virus or group thereof which includes, but is not limited to, gram-positive bacteria (e.g., Enterococcus sp. Group B streptococcus, Coagulase-negative staphylococcus sp. Streptococcus viridans, Staphylococcus aureus and saprophyicus, Lactobacillus and resistant strains thereof, for example); yeasts including, but not limited to, Candida albicans, for example; gram-negative bacteria such as, but not limited to, Escherichia coli, Klebsiella pneumoniae, Citrobacter koseri, Citrobacter freundii, Klebsiella oxytoca, Morganella morganii, Pseudomonas aeruginosa, Proteus mirabilis, Serratia marcescens, Diphtheroids (gnb), Rosebura, Eubacterium hallii, Faecalibacterium prauznitzli, Lactobacillus gasseria, Streptococcus mutans, Bacteroides thetaiotaomicron, Prevotella Intermedia, Porphyromonas gingivalis, Eubacterium rectale, Lactobacillus amylovorus, Bacillus subtilis, Bifidobacterium longum, Eubacterium rectale, E. eligens, E. dolichum, B. thetaiotaomicron, E. rectale, Actinobacteria, Proteobacteria, B. thetaiotaomicron, Bacteroides Eubacterium dolichum, Vulgatus, B. fragilis, bacterial phyla such as Firmicuties (Clostridia, Bacilli, Mollicutes), Fusobacteria, Actinobacteria, Cyanobacteria, Bacteroidetes, Archaea, Proteobacteria, and resistant strains thereof, for example; viruses such as, but not limited to, HIV, HPV, Flu, and MERSA, for example; and sexually transmitted diseases. In the case of detecting rare cell pathogens, a particle reagent is added that comprises a binding partner, which binds to the rare cell pathogen population. Additionally, for each population of cellular rare molecules on the pathogen, a reagent is added that comprises a binding partner for the cellular rare molecule, which binds to the cellular rare molecules in the population.

As mentioned above, some examples in accordance with the principles described herein are directed to methods of detecting a cell, which include natural and synthetic cells. The cells are usually from a biological sample that is suspected of containing target rare molecules, non-rare cells and rare cells. The samples may be biological samples or non-biological samples. Biological samples may be from a mammalian subject or a non-mammalian subject. Mammalian subjects may be, e.g., humans or other animal species.

### Kits for Conducting Methods

The apparatus and reagents for conducting a method in accordance with the principles described herein may be present in a kit useful for conveniently performing the method. In one embodiment, a kit comprises in packaged combination having modified affinity agent one for each different rare molecule acid to be isolated. The kit may also comprise one or more, cell affinity agent for cell containing the rare molecules, the porous matrix, optional capture particles, solution for spraying, filtering and reacting the mass labels, a droplet generators, capillaries nozzles for droplet formation, capillary channels for dilution, concentration or routing of solutions, droplets and molecules, solutions for forming droplets, and solutions for breaking droplets. The composition may contain labeled particles or capture particle entities, for example, as described above. Porous matrix, liquid holders and droplet generators can be in housing where the house can have vents, capillaries, chambers, liquid inlets and outlets. A solvent can be applied to droplet generators, liquid holders and porous matrix. The porous matrix can be remove-able.

Depending on method for analysis of rare molecules selected, reagents discussed in more detail herein below, may or may not be used to treat the samples during, prior or after the extraction of molecules from the rare cells and cell free samples.

The relative amounts of the various reagents in the kits can be varied widely to provide for concentrations of the reagents that substantially optimize the reactions that need to occur during the present methods and further to optimize substantially the sensitivity of the methods.

Under appropriate circumstances one or more of the reagents in the kit can be provided as a dry powder, usually lyophilized, including excipients, which on dissolution will provide for a reagent solution having the appropriate concentrations for performing a method in accordance with the principles described herein. The kit can further include a written description of a method utilizing reagents in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The spray solvent can be any spray solvent employed in electrospray mass spectroscopy. In some examples, solvents for electrospray ionization include, but are not limited to, polar organic compounds such as, e.g., alcohols (e.g., methanol, ethanol and propanol), acetonitrile, dichloromethane, dichloroethane, tetrahydrofuran, dimethylformamide, dimethyl sulphoxide, and nitromethane; non-polar organic compounds such as, e.g., hexane, toluene, cyclohexane; and water, for example, or combinations of two or more thereof. Optionally, the solvents may contain one or more of an acid or a base as a modifier (such as, volatile salts and buffer, e.g., ammonium acetate, ammonium bicarbonate, volatile acids such as formic acid, acetic acids or trifluoroacetic acid, heptafluorobutyric acid, sodium dodecyl sulphate, ethylenediamine tetraacetic acid, and non-volatile salts or buffers such as, e.g., chlorides and phosphates of sodium and potassium, for example.

In many examples, the sample is contacted with an aqueous phase prior to forming an emulsion. The aqueous phase may be solely water or may also contain organic solvents such as, for example, polar aprotic solvents such as, e.g., dimethylsulfoxide (DMSO), dimethyl-formamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume. In some examples, the pH for the aqueous medium is usually a moderate pH. In some examples, the pH of the aqueous medium is about 5 to about 8, or about 6 to about 8, or about 7 to about 8, or about 5 to about 7, or about 6 to about 7, or physiological pH. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE.

Cell and/or droplet lysis reagents are those that involve disruption of the integrity of the cellular membrane with a lytic agent, thereby releasing intracellular contents of the cells.

Numerous lytic agents are known in the art. Lytic agents that may be employed may be physical and/or chemical agents. Physical lytic agents include blending, grinding, and sonication, and combinations or two or more thereof, for example. Chemical lytic agents include, but are not limited to, non-ionic detergents, anionic detergents, amphoteric detergents, low ionic strength aqueous solutions (hypotonic solutions), bacterial agents, and antibodies that cause complement dependent lysis, and combinations of two or more thereof, for example, and combinations or two or more of the above. Non-ionic detergents that may be employed as the lytic agent include both synthetic detergents and natural detergents.

The nature and amount or concentration of lytic agent employed depends on the nature of the cells, the nature of the cellular contents, the nature of the analysis to be carried out, and the nature of the lytic agent, for example. The amount of the lytic agent is at least sufficient to cause lysis of cells to release contents of the cells. In some examples the amount of the lytic agent is (percentages are by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

Removal of lipids, platelets, and non rare cells may be carried out using, by way of illustration and not limitation, detergents, surfactants, solvents, and binding agents, and combinations of two or more of the above, for example, and combinations of two or more thereof. The use of a surfactant or a detergent as a lytic agent as discussed above accomplishes both cell lysis and removal of lipids. The amount of the agent for removing lipids is at least sufficient to remove at least about 50%, or at least about 60%, or at least about 70%, or at least about 80%, or at least about 90%, or at least about 95% of lipids from the cellular membrane. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example. In some examples, it may be desirable to remove or denature proteins from the cells, which may be accomplished using a proteolytic agent such as, but not limited to, proteases, heat, acids, phenols, and guanidinium salts, and combinations of two or more thereof, for example. The amount of the proteolytic agent is at least sufficient to degrade at least about 50%, or at least about 60%, or at least about 70%, or at least about 80%, or at least about 90%, or at least about 95% of proteins in the cells. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, samples are collected from the body of a subject into a suitable container such as, but not limited to, a cup, a bag, a bottle, capillary, or a needle, for example. Blood samples may be collected into VACUTAINER® containers, for example. The container may contain a collection medium into which the sample is delivered. The collection medium is usually a dry medium and may comprise an amount of platelet deactivation agent effective to achieve deactivation of platelets in the blood sample when mixed with the blood sample.

Platelet deactivation agents can be added to the sample such as, but are not limited to, chelating agents such as, for example, chelating agents that comprise a triacetic acid moiety or a salt thereof, a tetraacetic acid moiety or a salt thereof, a pentaacetic acid moiety or a salt thereof, or a hexaacetic acid moiety or a salt thereof. In some examples, the chelating agent is ethylene diamine tetraacetic acid (EDTA) and its salts or ethylene glycol tetraacetate (EGTA) and its salts. The effective amount of platelet deactivation agent is dependent on one or more of the nature of the platelet deactivation agent, the nature of the blood sample, level of platelet activation and ionic strength, for example. In some examples, for EDTA as the anti-platelet agent, the amount of dry EDTA in the container is that which will produce a concentration of about 1.0 to about 2.0 mg/mL of blood, or about 1.5 mg/mL of the blood. The amount of the platelet deactivation agent is that which is sufficient to achieve at least about 90%, or at least about 95%, or at least about 99% of platelet deactivation.

Moderate temperatures are normally employed, which may range from about 5° C. to about 70° C. or from about 15° C. to about 70° C. or from about 20° C. to about 45° C., for example. The time period for an incubation period is about 0.2 seconds to about 6 hours, or about 2 seconds to about 1 hour, or about 1 to about 5 minutes, for example. These temperature can be used to reverse fixations or other reactions.

An amount of aqueous medium employed is dependent on a number of factors such as, but not limited to, the nature and amount of the sample, the nature and amount of the reagents, the stability of rare cells, and the stability of rare molecules, for example. In some examples in accordance with the principles described herein, the amount of aqueous medium per 10 mL of sample is about 5 mL to about 100 mL, or about 5 mL to about 80 mL, or about 5 mL to about 60 mL, or about 5 mL to about 50 mL, or about 5 mL to about 30 mL, or about 5 mL to about 20 mL, or about 5 mL to about 10 mL, or about 10 mL to about 100 mL, or about 10 mL to about 80 mL, or about 10 mL to about 60 mL, or about 10 mL to about 50 mL, or about 10 mL to about 30 mL, or about 10 mL to about 20 mL, or about 20 mL to about 100 mL, or about 20 mL to about 80 mL, or about 20 mL to about 60 mL, or about 20 mL to about 50 mL, or about 20 mL to about 30 mL, for example.

Where one or more of the rare nucleic acids are part of a cell, the aqueous medium may also comprise a lysing agent for lysing of cells. A lysing agent is a compound or mixture of compounds that disrupt the integrity of the matrixs of cells thereby releasing intracellular contents of the cells. Examples of lysing agents include, but are not limited to, non-ionic detergents, anionic detergents, amphoteric detergents, low ionic strength aqueous solutions (hypotonic solutions), bacterial agents, aliphatic aldehydes, and antibodies that cause complement dependent lysis, for example. Various ancillary materials may be present in the dilution medium. All of the materials in the aqueous medium are present in a concentration or amount sufficient to achieve the desired effect or function.

In some examples, it may be desirable to fix the nucleic acids, proteins or cells of the sample. Fixation immobilizes the nucleic acids and preserves the nucleic acids structure and maintains the cells in a condition that closely resembles the cells in an in vivo-like condition and one in which the antigens of interest are able to be recognized by a specific affinity agent. The amount of fixative employed is that which preserves the nucleic acids or cells but does not lead to erroneous results in a subsequent assay. The amount of fixative depends on one or more of the nature of the fixative and the nature of the cells, for example. In some examples, the amount of fixative is about 0.05% to about 0.15% or about 0.05% to about 0.10%, or about 0.10% to about 0.15%, for example, by weight. Agents for carrying out fixation of the cells include, but are not limited to, cross-linking agents such as, for example, an aldehyde reagent (such as, e.g., formaldehyde, glutaraldehyde, and paraformaldehyde,); an alcohol (such as, e.g., C₁-C₅ alcohols such as methanol, ethanol and isopropanol); a ketone (such as a C₃-C₅ ketone such as acetone); for example. The designations C₁-C₅ or C₃-C₅ refer to the number of carbon atoms in the alcohol or ketone. One or more washing steps may be carried out on the fixed cells using a buffered aqueous medium.

In examples in which fixation is employed, extraction of nucleic acids can include a procedure for de-fixation prior to amplification. De-fixation may be accomplished employing, by way of illustration and not limitation, heat or chemicals capable of reversing cross-linking bonds, or a combination of both, for example.

In some examples utilizing the techniques, it may be necessary to subject the rare cells to permeabilization. Permeabilization provides access through the cell membrane to nucleic acids of interest. The amount of permeabilization agent employed is that which disrupts the cell membrane and permits access to the nucleic acids. The amount of permeabilization agent depends on one or more of the nature of the permeabilization agent and the nature and amount of the rare cells, for example. In some examples, the amount of permeabilization agent by weight is about 0.1% to about 0.5%, or about 0.1% to about 0.4%, or about 0.1% to about 0.3%, or about 0.1% to about 0.2%, or about 0.2% to about 0.5%, or about 0.2% to about 0.4%, or about 0.2% to about 0.3%, for example. Agents for carrying out permeabilization of the rare cells include, but are not limited to, an alcohol (such as, e.g., C₁-C₅ alcohols such as methanol and ethanol); a ketone (such as a C₃-C₅ ketone such as acetone); a detergent (such as, e.g., saponin, Triton® X-100, and Tween®-20); for example. One or more washing steps may be carried out on the permeabilized cells using a buffered aqueous medium.

The following examples further describe the specific embodiments of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis Mo.) unless otherwise noted. Abbreviations:
min=minute(s)
μm=micron(s)
mL=milliliter(s)
mg=milligrams(s)
μg=microgram(s)
w/w=weight to weight
RT=room temperature
hr=hour(s)
QS=quantity sufficient
Ab=antibody
mAb=monoclonal antibody
vol=volume
MW=molecular weight
wt=weight
Phosphate buffered saline (PBS)=3.2 mM Na₂HPO₄, 0.5 mM KH₂PO₄, 1.3 mM KCl, and 135 mM NaCl at pH 7.4
PBS-EDTA buffe=0.5M EDTA in PBS
NeutrAvidin=sulfhydryl-modified neutravidin in the range of 0.15-0.4 mg/mL
Mass label=
Capture particles=Magnetic beads BioMag® hydroxyl silica micro particles (46.2 mg/mL, 1.5 μm) with streptavidin (Bangs Lab Inc.)
Magnet =Dynal magnetic particle concentrator
Label particles=Silica amine label particle=Propylamine-functionalized silica nano-particles 200 μm, mesoporous pore sized 4 nm
Porous Matrix =WHATMAN® NUCLEOPORE™ Track Etch matrix, 25 mm diameter and 8.0 and 1.0 μM pore sizes

### Example 1

### Generation and Size Exclusion Filtration of Droplets

A group of cells of same type but different genotype are isolated. In this example it was a group of 10⁶ or more different antibody producing cells prepared by hybridomia techniques. In other examples, the group of cells were other rare cell types and the affinity agent was for a rare cell molecule. Preparation of cells with label particle with an antigen, in this case Bikunin protein (BBI Inc) and a fluorescent label, in this case Dylight 488. The compound library of 10⁶ different antibody producing hybridomia cells are bound to the labeled particle using bikinin as an affinity agent for immunoglobulin IgG molecules of interest. In other cases a cell cluster of antibody producing hybridomia cells are bound the molecules of interest. Alternative the cells can be labeled with fluorescent substrate for molecules of interest. Unbound affinity agent and/or fluorescent substrate, are washed away with and cells are mixed into an aqueous buffer with surfactant for droplet formation

A group of different of 10⁶ or more cDNA genes were isolated from the antibody producing cells (as above) for the variable kappa, gamma and lambda immunoglobulin domains. Additional unique B cell can be obtained by FACS sorting using antigen binding with fluourecent labels. Once isolated, the mRNA for variable kappa, gamma and lambda immunoglobulin domains are converted to a cDNA library by reverse transcriptase. In other samples, cell free RNA or DNA isolated from human blood can be used to generate a group of cDNA genes by cDNA converted by reverse transcriptase or DNA polymerase respectively. In some cases the, cDNA libray was captured onto capture particle with a nucleic acid affinity agent.

A group of different protein variations of insulin were isolated from the human blood using an capture particle and unique antibodies for insulin fragments. Unbound proteins were washed away using a magnet. The antibodies used were biotinylated. The variations of insulin were prepared for detection by treatment of capture particle with labeled particle which was a labeled nanoparticles (15 to 200 nm) with a release-able MS, in this case a peptide attached by a sulfidryl, and non-releasable fluorescent label, in this case Dylight 488 attached to NeutrAvidin. The labeled particle biotins are bound to capture particle NeutrAvidin and unbuound labeled particles are washed away. The compound library of 10⁶ different capture and labeled particles are bound and dissolved into an aqueous media.

Droplets were generated in the droplet generator (Bio-Rad QX100 system) containing the library of gene compounds or library of protein compounds or the library of cell compounds.

A method of removing the empty droplets but retaining contents of full droplets by size exclusion filtration droplet were diluted in PBS, and filtered through as filtration process as previously described in (Using Automated Microfluidic Filtration and Multiplex Immunoassay Magbanua M J M, Pugia M, Lee J S, Jabon M, Wang V, et al. (2015) A Novel Strategy for Detection and Enumeration of Circulating Rare Cell Populations in Metastatic Cancer Patients Using Automated Microfluidic Filtration and Multiplex Immunoassay. PLoS ONE 10(10)). The only change to the process was to use a vacuum filtration unit (Biotek Inc) for a standard ELISA plate fitted with the standard.

The sample was filtered through a membrane with 8.0 μm pores for the cell library and 1.0 μm pores for the protein library and 0.1 μm pores for the gene or 1.0 μm if captured on a particle. The cells in this library were ˜10 μm diameter (5 to 30 μm range), nucleic acids c DNA particle were ˜20 μm diameter (10 to 400 nm range), and protein capture with label particles were ˜1.5 μm diameter (1 to 2 μm range). Cell clusters were ˜75 μm average diameter, 50 to 300 μm range). Each droplet library contained 10⁴ to 10⁶ unique molecules in full droplets and 10⁶ to 10⁹ empty droplet.

Empty droplets were removed by filtration, sample on the porous matrix was subjected to a negative mBar, that is, a decrease greater than about −100 mBar from atmospheric pressure. The vacuum applied varied from −10 to −100 mBar during filtration. The droplets in a diluted sample was placed into the filtration station without mixing and the sample was filtered through the porous matrix. Stabilization of full and empty droplets was achieved as they not spill into the oil phase. After the oil phase was removed by vacuum filtration, a surfactant, in this case 0.5% Triton X 100 in PBS was added to destabilize the remaining droplets to spill the oil phase leaving content whether particles, cells or cDNA behind.

The cells retained were measured by fluorescent microscopy and demonstrated a recovery of >90%. The particles retained were measured by mass label release measurement by Mass Spectroscopy and demonstrated a recovery of >90%. The cDNA retained were measured by mass label release measurement by PCR and demonstrated a recovery of >90%. Overall this demonstrated a method allowing for retaining the contents of full droplets and removing the contents of empty droplets.

All patents, patent applications and publications cited in this application including all cited references in those patents, applications and publications, are hereby incorporated by reference in their entirety for all purposes to the same extent as if each individual patent, patent application or publication were so individually denoted.

While the many embodiments of the invention have been disclosed above and include presently preferred embodiments, many other embodiments and variations are possible within the scope of the present disclosure and in the appended claims that follow. Accordingly, the details of the preferred embodiments and examples provided are not to be construed as limiting. It is to be understood that the terms used herein are merely descriptive rather than limiting and that various changes, numerous equivalents may be made without departing from the spirit or scope of the claimed invention. All patents, patent applications and publications cited in this application including all cited references in those patents, applications and publications, are hereby incorporated by reference in their entirety for all purposes to the same extent as if each individual patent, patent application or publication were so individually denoted.

What is claimed is:

1. A method of generating droplets containing a library of compounds, said method comprising:
(a) generating a mixture of droplets containing said library of compounds and empty droplets in a water in oil emulsion; and
(b) removing the empty droplets from said emulsion by size exclusion filtration using a porous matrix.

2. The method of claim 1, wherein said empty droplets are destabilized into the oil phase.

3. The method of claim 2, wherein a destabilization agent is added to the immiscible liquid.

4. The method of claim 2, wherein the oil phase passes through the porous matrix.

5. The method of claim 1, wherein full droplets are stabilized to not spill into the oil phase.

6. The method of claim 5, wherein a stabilization agent is added to either immiscible liquid.

7. The method of claim 5, where retained contents remain in the droplets and do not pass through the porous matrix.

8. The method of claim 7, wherein the droplets are retained on the porous matrix.

9. The method of claim 1, wherein the retaining contents are particles, cells and molecules.

10. The method of claim 9, where particles, cells and molecules are retained on the porous matrix.

11. The method of claim 9, wherein retained contents are released from droplets but do not passes through the porous matrix.

12. The method of claim 11, wherein retaining contents are particles, cells and molecules captured onto the porous matrix.

13. The method of claim 11, wherein the contents are retained on the porous matrix.

14. The method of claim 1, wherein droplets are produced rapidly.

15. The method of claim 1, wherein the droplets contain an excess of empty droplets.

16. The method of claim 1, wherein the library of compounds is selected from the group consisting of organic molecules, biochemicals, particulates, cells, or macromolecules which contain unique group members.


SEQUENCE LISTING (18 sequences)

SEQ ID NO: 1 - protein, 19 aa, Artificial
  Synthetic
MALWMRLLPLLALLALWGP

SEQ ID NO: 2 - protein, 10 aa, Artificial
  Synthetic
MALWMRLLPL

SEQ ID NO: 3 - protein, 9 aa, Artificial
  Synthetic
ALLALWGPD

SEQ ID NO: 4 - protein, 34 aa, Artificial
  Synthetic
AAAFVNQHLCGSHLVEALYLVCGERGFFYTPKTR

SEQ ID NO: 5 - protein, 23 aa, Artificial
  Synthetic
PAAAFVNQHLCGSHLVEALYLVC

SEQ ID NO: 6 - protein, 13 aa, Artificial
  Synthetic
PAAAFVNQHLCGS

SEQ ID NO: 7 - protein, 12 aa, Artificial
  Synthetic
CGSHLVEALYLV

SEQ ID NO: 8 - protein, 8 aa, Artificial
  Synthetic
VEALYLVC

SEQ ID NO: 9 - protein, 8 aa, Artificial
  Synthetic
LVCGERGF

SEQ ID NO: 10 - protein, 6 aa, Artificial
  Synthetic
FFYTPK

SEQ ID NO: 11 - protein, 31 aa, Artificial
  Synthetic
REAEDLQVGQVELGGGPGAGSLQPLALEGSL

SEQ ID NO: 12 - protein, 12 aa, Artificial
  Synthetic
REAEDLQVGQVE

SEQ ID NO: 13 - protein, 8 aa, Artificial
  Synthetic
LGGGPGAG

SEQ ID NO: 14 - protein, 11 aa, Artificial
  Synthetic
SLQPLALEGSL

SEQ ID NO: 15 - protein, 21 aa, Artificial
  Synthetic
GIVEQCCTSICSLYQLENYCN

SEQ ID NO: 16 - protein, 14 aa, Artificial
  Synthetic
GIVEQCCTSICSLY

SEQ ID NO: 17 - protein, 7 aa, Artificial
  Synthetic
QLENYCN

SEQ ID NO: 18 - protein, 7 aa, Artificial
  Synthetic
CSLYQLE